(19) United States
(12) Patent Application Publication
(10) Pub. No.: US 2020/0297775 A1
(43) Pub. Date: Sep. 24, 2020

(54) ENHANCED DIRECT CARDIAC REPROGRAMMING

(71) Applicant: The J. David Gladstone Institutes, A Testamentary Trust Established Under the Will of J. David Glads, San Francisco, CA (US)

(72) Inventors: Deepak SRIVASTAVA, San Francisco, CA (US); Tamer MOHAMED, San Francisco, CA (US)

(21) Appl. No.: 16/088,764

(22) PCT Filed: Mar. 30, 2017
(86) PCT No.: PCT/US17/25132

§ 371 Date: Sep. 26, 2018

Related U.S. Application Data
(60) Provisional application No. 62/315,437, filed on Mar. 30, 2016.
(60) Provisional application No. 62/370,344, filed on Aug. 3, 2016.

(51) Int. Cl. A61K 35/34; C12N 15/113; A61K 38/17; A61K 45/06; A61K 31/573; A61P 9/00

(52) U.S. Cl. CPC A61K 35/34; C12N 15/1136; C12N 15/1138; A61P 9/00; A61K 45/06; A61K 31/573; A61K 38/1709

(57) ABSTRACT

The present disclosure provides methods for generating induced cardiomyocytes. The present disclosure further provides methods and compositions for treating cardiovascular disease.

### CROSS REFERENCE TO RELATED APPLICATIONS

This application claims priority to U.S. Provisional Application 62/315,437, filed Mar. 30, 2016, and U.S. Provisional Application 62/370,344, filed Aug. 3, 2016, which are hereby incorporated by reference in their entirety for all purposes.

### NIH FUNDING STATEMENT

This invention was made with government support under Grant HL100406 awarded by the National Institutes of Health. The government has certain rights in the invention.

### FIELD OF THE INVENTION

The present invention relates generally to the fields of cell reprogramming and cell differentiation.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

A myocardial infarction (MI), or heart attack, is caused by the blockage of blood flow in the heart, which reduces oxygen levels, damages tissues (ischemia), and can kill nearly one billion cardiomyocytes. Burridge et al. (2012) Cell Stem Cell 10:16-28.

Following a MI, fibroblasts migrate into the infarcted area and proliferate, creating a cardiomyocyte-depleted scar that cannot contribute to the electrophysiologically driven contractions of the heart. Heart failure often ensues, leading to fatigue, peripheral edema, and even death.

Since the first successful generation of induced cardiomyocyte-like mouse cells (iCMs), combinations of genes have been identified that can be used to induce or enhance cardiac reprogramming in mammalian cells. Ieda et al. (2010) Cell 142:375-386; Christoforou et al. (2013) PLoS ONE 8:e63577; Addis et al. (2013) J. Mol. Cell Cardiol. 60:97-106; Jayawardena et al. (2012) Circ. Res. 110:1465-1473; Nam Y et al., PNAS USA. 2013; 110:5588-5593; Wada R et al. PNAS USA. 2013; 110:12667-12672; and Fu J et al., Stem Cell Reports. 2013; 1:235-247. The reprogramming has been based on the use of certain core transcription factors (TFs), including factors Gata4, Mef2C and Tbx5, Hand2, Nkx2.5, Myocardin, SRF, Mesp1, or miR-133 in the original reprogramming cocktail. More recently, there have been some slight improvements in the quality and efficiency of iCMs generated in vitro. Wang et al. (2015) Circ. Res. 116:237-24.

In vivo studies have also produced reprogrammed iCMs. For example, introducing cardiac reprogramming cocktails into injured murine hearts in vivo produced reprogrammed iCMs, increased heart function, and decreased scar size after injury. However, cardiac function was not completely restored. Jayawardena et al. (2012) Circ. Res. 110:1465-1473; Qian et al (2012) Nature 485:593-598. Cardiac function and scar size did improve with Gata4, Mef2C and Tbx5 and was further enhanced with Thymosin β4 and VEGF. Bock-Marquette et al. (2004) Nature 432:466-472; Mathison et al. (2012) J. Am. Heart Assoc. 1:e005652.

Despite tremendous efforts, effective therapies for heart failure remain elusive. There remains a need for improved reprogramming and cardiac cell-fate conversion, which will lead to the development of better therapeutic approaches for treating cardiac conditions. The present invention addresses this need.

### SUMMARY OF THE INVENTION

The present invention provides improved methods for inducing non-cardiomyocyte mammalian cells into, e.g., cardiomyocytes or cardio-myocyte-like cells. In certain embodiments, the invention provides methods for inducing human cells into human cardiac cells, e.g., human cardiomyocytes. The present invention provides the use of a combination of reprogramming factors and introduced agents that allow efficient generation of induced cardiomyocytes in vitro, in situ and/or in vivo.

In one aspect, the invention provides methods for generating an induced cardiomyocyte, comprising administering an effective amount of one or more reprogramming factors and one or more agents to a non-cardiomyocyte, thereby generating an induced cardiomyocyte or a cardiomyocyte-like cell. The methods of administering reprogramming factors and agents can be performed in vitro or in vivo to generate an induced cardiomyocyte.

In specific aspects, the invention provides methods for generating an induced cardiomyocyte, comprising administering to a non-cardiomyocyte an effective amount of an agent that inhibits WNT activity. In other specific aspects, the invention provides methods for generating an induced cardiomyocyte, comprising administering to a non-cardiomyocyte an effective amount of an agent that inhibits WNT activity and at least one reprogramming factor. In yet other specific aspects, the invention provides methods for generating an induced cardiomyocyte, comprising administering to a non-cardiomyocyte an effective amount of an agent that inhibits WNT activity and an anti-inflammatory agent. In related aspects, methods are provided for administering to a subject an agent that inhibits WNT activity for generating an induced cardiomyocyte in vivo. Non-limiting examples of such agents include small molecule inhibitors of WNT activity and siRNA inhibitors of WNT activity, as described more fully herein.

In other aspects, the invention provides methods for generating an induced cardiomyocyte, comprising administering to a non-cardiomyocyte an effective amount of an agent that inhibits TGF-β activity. In other specific aspects, the invention provides methods for generating an induced cardiomyocyte, comprising administering to a non-cardiomyocyte an effective amount of an agent that inhibits TGF-β activity and at least one reprogramming factor. In yet other specific aspects, the invention provides methods for generating an induced cardiomyocyte, comprising administering to a non-cardiomyocyte an effective amount of an agent that inhibits TGF-β activity and an anti-inflammatory agent. In related aspects, methods are provided for administering to a subject an agent that inhibits TGF-β activity for generating an induced cardiomyocyte in vivo. Non-limiting examples of such agents include small molecule inhibitors of TGF-β activity and siRNA inhibitors of TGF-β activity, as described more fully herein.

In certain aspects, the invention provides a method for generating an induced cardiomyocyte, the method comprising: administering an effective amount of a WNT inhibitor, an effective amount of a TGF-β inhibitor, and one or more reprogramming factors, thereby generating an induced cardiomyocyte or a cardiomyocyte-like cell.

Exemplary reprogramming factors for use in the invention include, e.g., Baf60c, Esrrg, Gata4, Gata6, Hand2, Irx4, Isl1, Mef2c, Mesp1, Mesp2, Myocardin, Nkx2.5, SRF, Tbx5, Tbx20, Zfpm2, miR-133, or any combination thereof.

In certain aspects, the WNT inhibitor and/or the TGF-β inhibitor is a small molecule. In other certain aspects, the WNT inhibitor and/or the TGF-β inhibitor is an siRNA that inhibits WNT or TGF-β signaling activity, respectively. When both a WNT inhibitor and a TGF-β inhibitor are administered, the TGF-β inhibitor can be administered prior to administration of the WNT inhibitor, or concurrently with the administration of the WNT inhibitor.

The non-cardiomyocyte used can be any cell amenable to induction into a cardiomyocyte. Non-limiting examples include a somatic cell, a cardiac fibroblast, a non-cardiac fibroblast, a cardiac progenitor cell, and a stem cell. In specific aspects, the non-cardiomyocyte is a human cell.

In specific embodiments, the reprogramming factors administered to the non-cardiomyocyte include Gata4, Mef2c, and Tbx5 (i.e., GMT). In other specific embodiments, the reprogramming factors administered to the non-cardiomyocyte include Myocardin, Mef2c, and Tbx5 (i.e., MMT). In yet other specific embodiments, the reprogramming factors administered to the non-cardiomyocyte include Gata4, Mef2c, Tbx5, and Myocardin (i.e., 4F). In other embodiments, the reprogramming factors include Gata4, Mef2c, and Tbx5, Essrg, Myocardin, Zfpm2, and Mesp1 (i.e., 7F).

Accordingly, in certain embodiments, the invention provides methods for generating an induced cardiomyocyte, the method comprising administering to a non-cardiomyocyte an effective amount of a WNT inhibitor, an effective amount of a TGF-β inhibitor, and reprogramming factors comprising Gata4, Mef2c, and Tbx5 (i.e. “GMT”. In other embodiments, the invention provides methods for generating an induced cardiomyocyte, the method comprising administering to a non-cardiomyocyte an effective amount of a WNT inhibitor, an effective amount of a TGF-β inhibitor, and reprogramming factors comprising Myocardin, Mef2c, and Tbx5 (i.e. “MMT”). These methods may also include the administration of an anti-inflammatory agent. These methods can be carried out in vitro or in vivo.

The TGF-β inhibitor and the WNT inhibitor may be administered to a subject simultaneously or sequentially, and may also be administered simultaneously or sequentially with the one or more reprogramming factors and/or anti-inflammatory agents. In a more specific aspect, methods are provided for generating an induced cardiomyocyte by administering to a non-cardiomyocyte an effective amount of a TGF-β inhibitor for a first period of time prior to the addition of a WNT inhibitor. In another specific aspect, methods are provided for generating an induced cardiomyocyte by administering to a non-cardiomyocyte an effective amount of a TGF-β inhibitor simultaneously with the addition of a WNT inhibitor.

In specific aspects, methods are provided for generating an induced cardiomyocyte by culturing a non-cardiomyocyte in vitro with an effective amount of a TGF-β inhibitor and an effective amount of a WNT inhibitor in combination with Gata4, Mef2C and Tbx5. In other specific aspects, methods are provided for generating an induced cardiomyocyte by culturing a non-cardiomyocyte in vitro with an effective amount of a TGF-β inhibitor and an effective amount of a WNT inhibitor in combination with Myocardin, Mef2C and Tbx5.

In specific aspects, the methods use reprogramming factors including Gata4, Mef2c, and Tbx5. In other specific aspects, the methods use reprogramming factors including Myocardin, Mef2c, and Tbx5. The methods also may further comprise administering to the subject an effective amount of an anti-inflammatory molecule, including a steroidal anti-inflammatory such as corticosteroid (e.g., dexamethasone) or a non-steroidal anti-inflammatory drug (NSAID).

In addition to the use of a WNT inhibitor, a TGF-β inhibitor, and one or more reprogramming factors, a corticosteroid (e.g., dexamethasone) can be administered to the non-cardiomyocyte to increase efficiency of the induction of the cell to a cardiomyocyte or cardiomyocyte-like cell.

The present invention provides various methods for treating a cardiovascular disease. In certain aspects, the disclosure provides methods of treating a cardiovascular disease comprising administering to a subject in need thereof an effective amount of a WNT inhibitor, thereby generating an induced cardiomyocyte or a cardiomyocyte-like cell. In other aspects, the disclosure provides methods of treating a cardiovascular disease comprising administering to a subject in need thereof an effective amount of a TGF-β inhibitor, thereby generating an induced cardiomyocyte or a cardiomyocyte-like cell. In a preferred aspect, the disclosure provides methods of treating a cardiovascular disease comprising administering to a subject in need thereof an effective amount of a WNT inhibitor and an effective amount of a TGF-β inhibitor, thereby generating an induced cardiomyocyte or a cardiomyocyte-like cell.

Accordingly, in another specific embodiment, the invention provides methods for treating a cardiovascular disease comprising administering to a subject in need thereof an effective amount of a WNT inhibitor, an effective amount of a TGF-β inhibitor, and one or more reprogramming factors. In certain aspects of the invention, the WNT inhibitor and/or the TGF-β inhibitor can be small molecules. In other aspects of the invention, the WNT inhibitor and/or the TGF-β inhibitor can be siRNA molecules. In certain aspects of the invention, the reprogramming factors that can be administered include, e.g., Baf60c, Esrrg, Gata4, Gata6, Hand2, Irx4, Isl1, Mef2c, Mesp1, Mesp2, Myocardin, Nkx2.5, SRF, Tbx5, Tbx20, Zfpm2, miR-133, or any combination thereof. In preferred aspects, the methods are provided for generating an induced cardiomyocyte by administering to a non-cardiomyocyte an effective amount of a TGF-β inhibitor and an effective amount of a WNT inhibitor in combination with administration of other combinations of reprogramming factors, e.g., Baf60c, Esrrg, Gata4, Gata6, Hand2, Irx4, Isl1, Mef2c, Mesp1, Mesp2, Myocardin, Nkx2.5, SRF, Tbx5, Tbx20, Zfpm2, miR-133, or any combination thereof.

In specific aspects, the methods of treating a cardiovascular disease comprise administering to a subject in need thereof an effective amount of a WNT inhibitor and an effective amount of a TGF-β inhibitor with the reprogramming cocktail GMT to generate an induced cardiomyocyte or a cardiomyocyte-like cell in vivo in a subject. In other specific aspects, the methods of treating a cardiovascular disease comprise administering to a subject in need thereof an effective amount of a WNT inhibitor and an effective amount of a TGF-β inhibitor with the reprogramming cocktail MMT to generate an induced cardiomyocyte or a cardiomyocyte-like cell in vivo in a subject.

In other embodiments, the invention provides methods of treating a cardiovascular disease comprising administering to a subject in need thereof an effective amount of an induced cardiomyocyte produced by the methods described herein. In yet another aspect, the disclosure provides compositions comprising a population of the isolated induced cardiomyocytes described herein and a carrier, optionally a pharmaceutically acceptable excipient. In some embodiments, the compositions further comprise a stabilizer and/or a preservative.

In some embodiments, the non-cardiomyocyte is cultured for a period of time in the presence of the TGF-β inhibitor prior to the addition of the WNT inhibitor, for example, between about 6 hours and about 72 hours prior to addition of the WNT inhibitor. In one preferred embodiment, the non-cardiomyocyte is cultured in the presence of the TGF-β inhibitor for about 24 hours prior to addition of the WNT inhibitor. In some embodiments, the non-cardiomyocyte is cultured in the presence of the TGF-β inhibitor and the WNT inhibitor concurrently for a period of time.

In some embodiments, the invention provides isolated induced cardiomyocytes generated according to the methods of the invention, wherein the induced cardiomyocyte expresses at least one cardiac gene at a higher level or a lower level than a naturally occurring cardiomyocyte. In certain aspects, a substantially homogenous population of induced cardiomyocytes is generated. In some embodiments, the induced cardiomyocytes of the substantially homogenous population express at least one cardiac gene at a higher level or a lower level than a naturally occurring cardiomyocyte.

Certain aspects of the invention include the production of a composition comprising a population of isolated induced cardiomyocytes. Such compositions may further comprise, e.g., a carrier, a pharmaceutically acceptable excipient, a stabilizer and/or a preservative. In specific aspects, the composition comprises a population of isolated induced cardiomyocytes that is substantially homogenous.

In some embodiments, the methods comprise administering a TGF-β activator to the cell subsequent to the administration of the TGF-β inhibitor to the non-cardiomyocyte.

In some embodiments, the non-cardiomyocyte is selected from the group consisting of a somatic cell, a cardiac fibroblast, a non-cardiac fibroblast, a cardiac progenitor cell, and a stem cell. In some embodiments, the non-cardiomyocyte is a mammalian non-cardiomyocyte. In preferred embodiments, the non-cardiomyocyte is a human non-cardiomyocyte.

In some embodiments, the non-cardiomyocyte is first contacted with a TGF-β inhibitor at between about 12 hours and about 36 hours after the reprogramming factors have been introduced. In other embodiments, the non-cardiomyocyte is first contacted with a WNT inhibitor at between about 24 hours and about 72 hours after the reprogramming factors have been introduced.

In some embodiments, the WNT inhibitor is selected from the group consisting of XAV939, D4476, IWR1, and myricetin. In other embodiments the WNT inhibitor is siRNA against WNT or a member of the WNT signaling pathway.

In some embodiments, the TGF-β inhibitor is selected from the group consisting of SB431542, D4476, LDN-193189, dexamethasone, and LY364947. In other embodiments the TGF-β inhibitor is siRNA against TGF-β or a member of the TGF-β signaling pathway.

In some embodiments, the WNT inhibitor and/or the TGF-β inhibitor are siRNA molecules that inhibit the activity of the WNT and/or TGF-β pathways, respectively.

In some embodiments, the WNT inhibitor, the TGF-β inhibitor, or both are removed after day 10 of reprogramming.

In some embodiments, the TGF-β inhibitor is administered to the subject for a period of time prior to the administration of the WNT inhibitor, for example, between about 6 hours and about 72 hours prior to administration of the WNT inhibitor. In one preferred embodiment, the subject is administered the TGF-β inhibitor for about 24 hours prior to administration of the WNT inhibitor. In some embodiments, the subject is administered the TGF-β inhibitor and the WNT inhibitor concurrently for a period of time.

In some embodiments, a TGF-β inhibitor is administered to the subject at between about 12 hours and about 36 hours after the reprogramming factor has been introduced. In other embodiments, a WNT inhibitor is administered to the subject at between about 24 hours and about 72 hours after the reprogramming factor has been introduced.

These aspects and other features and advantages of the invention are described below in more detail. Those skilled in the art will recognize, or be able to ascertain using no more than routine experimentation, many equivalents to the specific embodiments of the invention described herein. Such equivalents are intended to be encompassed by the following disclosure and claims.

### BRIEF DESCRIPTION OF THE FIGURES

FIG. 1 is a schematic for the drug-screening strategy to demonstrate WNT and TGF-β signaling barriers in direct cardiac reprogramming using an α-MHC-GFP reporter in Thy1⁺ mouse cardiac fibroblasts.

FIG. 2 is a Z-score plot for the 5500 compounds identified using the drug screening strategy of FIG. 1, showing the top hits with a Z-score over 5.

FIG. 3 is a bar graph showing the efficiency of the validated hits using the drug screening strategy of FIG. 1, seven of which inhibit TGF-β or WNT (n=3).

FIG. 4 is a bar graph showing the quantification of the effect of different dosages of SB431542 on reprogramming efficiency of GMT (n=2 independent experiments, *p<0.05).

FIG. 5 is a bar graph showing the quantification of the effect of addition of SB431542 at different time points on reprogramming efficiency of GMT (n=2 independent experiments, *p<0.05).

FIG. 6 is a bar graph showing the quantification of the effect of addition of XAV939 at different time points on reprogramming efficiency of GMT (n=2 independent experiments, *p<0.05).

FIG. 7 is a bar graphs showing the quantification of the effect of different dosages of XAV939 on reprogramming efficiency of GMT either alone or in the presence or absence of SB431542 (n=2 independent experiments, *p<0.05).

FIG. 8 is a bar graphs showing the quantification of the effect of different dosages of XAV939 on reprogramming efficiency of GMT with SB431542 added on day 1 (n=2 independent experiments, *p<0.05).

FIG. 9 is a bar graph showing a time course in which SB431542 and XAV939 were removed from GMTc transduced fibroblasts on the indicated day of reprogramming with GMT.

FIG. 10 are representative FACS plots for GFP⁺ iCMs after two weeks of reprogramming with TGF-β inhibitor SB431542 and WNT inhibitor XAV939, using an αMHC-GFP as a reporter for reprogramming.

FIG. 11 shows quantification of the percentage of iCMs that exhibits spontaneous calcium transients over 8 weeks of reprogramming (n=200 cells analyzed at each time point from two independent experiments, *p<0.05 compared to GMT).

FIG. 12 is a Principal Component Analysis plot showing that GMT-reprogrammed fibroblasts are at an intermediate state between fibroblasts and cardiomyocytes.

FIG. 13 is a bar graph showing the top differentially expressed genes between GMT and GMTc iCMs at 5 weeks.

FIG. 14 is a bar graph showing that the excess TGFβ1 (TGF-β) ligand introduced during reprogramming reversed the effect of SB431542 (TGFβi); BMP4 and activin did not have a significant effect.

FIG. 15 is a bar graph showing that overexpression of constitutively active SMAD2 or SMAD3 abolished enhanced cardiac reprogramming by SB431542 (TGFβi) (n=3, *p<0.05).

FIG. 16 is a bar graph showing that knocking down either ALK4 or ALK5 receptors enhanced cardiac reprogramming similar to that of SB431542 (TGFβi), without affecting the efficiency of reprogramming with SB431542 (n=3,*p<0.05).

FIG. 17 is a bar graph showing that the glycogen synthase kinase 3 beta (GSK3β) inhibitor CHIR99021 largely reversed the effects of SB431542 (TGFβi) and XAV939 (WNTi); activating the noncanonical WNT pathway through WNT5 or WNT11 only had an insignificant effect (n=3, *p<0.05).

FIG. 18 is a line graph showing that SB431542 and XAV939 enhance in vivo reprogramming with GMT as evidenced by changes in ejection fraction (ΔEF) as assessed by echocardiography during the experiment at 1, 2, 4, 8 and 12 weeks.

FIG. 19 is a series of bar graphs showing that stroke volume (SV), ejection fraction (EF), cardiac output (CO), and scar size were significantly improved in GMTc-treated mice compared to GMT-treated and control mice (dsRed or dsRedc) FIG. 20 is a schematic of lineage tracing using ROSA-YFP/Periostin Cre mice, to track the cell fate conversion of fibroblasts into cardiomyocytes.

FIG. 21 is a bar graph showing quantification of the fold increase in in vivo reprogrammed iCM number in multiple heart sections using GMT and GMTc (n=5 animals in each group, *p<0.05).

FIG. 22 is a Principal Component Analysis (PCA) plot for the global transcriptome of fibroblasts, neonatal mouse cardiomyocytes (CM), GMT iCMs in vivo (GMT), GMTc iCMs in vivo (GMTc), and adult ventricular cardiomyocytes assessed by RNA-seq.

FIG. 23 is a bar graph for the top GO terms for the differentially expressed genes between in vivo GMT and GMTc iCMs.

FIG. 24 is schematic representation of the strategy for generating a cell line of human cardiac fibroblasts using Floxed T-Antigen.

FIG. 25 shows representative FACS plots and quantification shows the efficiency of adult human cardiac fibroblast reprogramming with SB431542 and XAV939 using TNT-GCaMP as a reporter and αMHC antibody staining.

FIG. 26 shows spontaneous calcium transients within 3 weeks of reprogramming (top panel) and quantification of the percentage of cells that exhibited spontaneous calcium transients at 2, 4, 6, and 8 weeks of reprogramming (bottom panel) (n=100 cell in each group, *p<0.05).

FIG. 27 shows a Principal Component Analysis (PCA) plot for full gene expression profile from RNAseq of human cardiac fibroblasts, 7F reprogrammed iCM, or 7Fc reprogrammed iCMs.

FIG. 28 is a bar graph for the top Gene Ontology term (GO) annotation for the differentially expressed genes between in vivo 7F iCMs and 7Fc iCMs.

FIG. 29 shows representative FACS plots demonstrating that human fibroblast reprogramming occurred with SB431542 and XAV939 and four factors (Gata4, Mef2c, Tbx5, and Myocardin) (4Fc).

FIG. 30 is a bar graph demonstrating that human fibroblast reprogramming occurred with SB431542 and XAV939 and four factors (Gata4, Mef2c, Tbx5, and Myocardin) (4Fc).

FIG. 31 is a bar graph showing quantification of the fold increase in in vivo reprogrammed iCM number in multiple heart sections using GMT and GMTc (n=5 animals in each group, *p<0.05).

FIG. 32 is a bar graph showing the efficiency of MMT reprogramming using siRNA molecules as either a WNT inhibitor, a TGF-β inhibitor or both.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (2002) Cold Spring Harbor Laboratory Press; Sohail (2004) Gene Silencing by RNA Interference: Technology and Application (CRC Press); Sell (2013) Stem Cells Handbook.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cardiomyocyte” includes a plurality of cardiomyocytes.

### Definitions

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

“Administration,” “administering” and the like, when used in connection with a composition of the invention refer both to direct administration, which may be administration to non-cardiomyocytes in vitro, administration to non-cardiomyocytes in vivo, administration to a subject by a medical professional or by self-administration by the subject and/or to indirect administration, which may be the act of prescribing a composition of the invention. When used herein in reference to a cell, refers to introducing a composition to the cell. Typically, an effective amount is administered, which amount can be determined by one of skill in the art. Any method of administration may be used. Small molecules may be administered to the cells by, for example, addition of the small molecules to the cell culture media or injection in vivo to site of cardiac injury. Administration to a subject can be achieved by, for example, intravascular injection, intramyocardial delivery, and the like.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac α-actinin. The term “cardiomyocyte” as used herein is an umbrella term comprising any cardiomyocyte subpopulation or cardiomyocyte subtype, e.g., atrial, ventricular and pacemaker cardiomyocytes.

The term “cardiomyocyte-like cells” is intended to mean cells sharing features with cardiomyocytes, but which may not share all features. For example, a cardiomyocyte-like cell may differ from a cardiomyocyte in expression of certain cardiac genes.

The term “culture” or “cell culture” means the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells. Culture medium may be characterized by functional properties such as, but not limited to, the ability to maintain cells in a particular state (e.g., a pluripotent state, a quiescent state, etc.), to mature cells—in some instances, specifically, to promote the differentiation of progenitor cells into cells of a particular lineage (e.g., a cardiomyocyte).

As used herein the term “effective amount” in reference to a composition of a WNT inhibitor, a TGF-β inhibitor, or combinations thereof is an amount that is sufficient to generate an induced cardiomyocyte. The non-cardiomyocytes are contacted with an amount of the composition of a WNT inhibitor, a TGF-β inhibitor, or combinations thereof effective to generate an induced cardiomyocyte. When used herein in reference to administration to a subject in need thereof, the terms “amount effective” or “effective amount” mean an amount of a composition of a WNT inhibitor, a TGF-β inhibitor, or combinations thereof or induced cardiomyocytes which treat a cardiovascular disease. An effective amount can be administered in one or more administrations, applications or dosages. Such delivery is dependent on a number of variables including the time period for which the individual dosage unit is to be used, the bioavailability of the composition, the route of administration, etc. It is understood, however, that specific amounts of the compositions (e.g., a composition of a WNT inhibitor, a TGF-β inhibitor, or combinations thereof or induced cardiomyocytes) for any particular subject depends upon a variety of factors including the activity of the specific agent employed, the age, body weight, general health, sex, and diet of the subject, the time of administration, the rate of excretion, the composition combination, severity of the particular cardiovascular disease being treated and form of administration.

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. Further, the expression level of multiple genes can be determined to establish an expression profile for a particular sample. As used herein, the term “lower level” in reference to expression level refers to an amount in an induced cardiomyocyte that is less than the amount in a naturally occurring cardiomyocyte control sample. The term “higher level” in reference to expression level refers to an amount in an induced cardiomyocyte that is less than the amount in a naturally occurring cardiomyocyte control sample.

The term “induced cardiomyocyte” refers to a non-cardiomyocyte (and its progeny) that has been transformed into a cardiomyocyte (and/or cardiomyocyte-like cell). The methods of the present disclosure can be used in conjunction with any methods now known or later discovered for generating induced cardiomyocytes, for example, to enhance other techniques. For example, the compositions of a WNT inhibitor, TGF-β inhibitor, or combinations thereof can be used in conjunction with direct reprogramming techniques.

The term “inhibitor” as used herein refers to an agent with the ability to inhibit the expression, function, activity, etc. of a target molecule or signaling pathway. Inhibitors of the invention include, but are not limited to, small molecules, siRNA, antisense RNA, proteins, peptides, aptamers, antibodies and fragments thereof.

As used herein the term “isolated” with reference to a cell, refers to a cell that is in an environment different from that in which the cell naturally occurs, e.g., where the cell naturally occurs in a multicellular organism, and the cell is removed from the multicellular organism, the cell is “isolated.” For example, an isolated cell is a cell that is separated form tissue or cells of dissimilar phenotype or genotype.

The term “non-cardiomyocyte” as used herein refers to any cell or population of cells in a cell preparation not fulfilling the criteria of a “cardiomyocyte” as defined and used herein. Non-limiting examples of non-cardiomyocytes include somatic cells, cardiac fibroblasts, non-cardiac fibroblasts, cardiac progenitor cells, and stem cells.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

The terms “regenerate,” “regeneration” and the like as used herein in the context of injured cardiac tissue shall be given their ordinary meanings and shall also refer to the process of growing and/or developing new cardiac tissue in a heart or cardiac tissue that has been injured, for example, injured due to ischemia, infarction, reperfusion, or other disease. In some embodiments, cardiac tissue regeneration comprises generation of cardiomyocytes.

As used herein “reprogramming” includes transdifferentiation, dedifferentiation and the like.

As used herein, the term “reprogramming efficiency” refers to the number of cells in a sample that are successfully reprogrammed to cardiomyocytes relative to the total number of cells in the sample. Reprogramming efficiency may be measured as a function of cardiomyocyte markers. Such pluripotency markers include, but are not limited to, the expression of cardiomyocyte marker proteins and mRNA, cardiomyocyte morphology and electrophysiological phenotype. Non-limiting examples of cardiomyocyte markers include, α-sarcoglycan, atrial natriuretic peptide (ANP), bone morphogenetic protein 4 (BMP4), connexin 37, connexin 40, crypto, desmin, GATA4, GATA6, MEF2C, MYH6, myosin heavy chain, NKX2.5, TBX5, and Troponin T. In some aspects, reprogramming efficiency is increased by about 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to a control. Non-limiting examples of appropriate controls include a sample that has not been exposed to an effective amount of a WNT inhibitor, TGF-β inhibitor, with or without reprogramming factors, or any combination thereof.

The term “reprogramming factor” as used herein includes a factor that is introduced for expression in a cell to assist in the reprogramming of the cell into an induced cardiomyocyte. Reprogramming factors include, but are not limited to, transcription factors.

The term “stem cells” refer to cells that have the capacity to self-renew and to generate differentiated progeny. The term “pluripotent stem cells” refers to stem cells that can give rise to cells of all three germ layers (endoderm, mesoderm and ectoderm), but do not have the capacity to give rise to a complete organism.

As used herein the term “subject” refers to a mammal, preferably a human, but includes and is not limited to non-human primates, murines (i.e., mice and rats), canines, felines, equines, bovines, ovines, porcines, caprines, etc. In some embodiments, the subject is a human subject. The terms “subject” and “patient” may be used herein interchangeably.

The terms “transforming growth factor beta”, “TGF-β” and their equivalents refer to any of the TGFβ secreted proteins belonging to the subfamily of the transforming growth factor β (TGFβ) superfamily. TGFβs (TGFβ1, TGFβ2, TGFβ3) are multifunctional peptides that regulate proliferation, differentiation, adhesion, and migration and in many cell types. The mature peptides may be found as homodimers or as heterodimers with other TGFβ family members. TGFβs interact with transforming growth factor beta receptors (TGF-β Rs, or TGFβRs) on the cell surface, which binding activates MAP kinase-, Akt-, Rho- and Rac/cdc42-directed signal transduction pathways, the reorganization of the cellular architecture and nuclear localization of SMAD proteins, and the modulation of target gene transcription. Inhibitors of TGFβ signaling, can be readily be identified by one of ordinary skill in the art by any of a number of methods, for example competitive binding assays for binding to TGFβ or TGFβ receptors, or functional assays, e.g. measuring suppression of activity of downstream signaling proteins such as MAPK, Akt, Rho, Rac, and SMADs, e.g., AR-Smad, etc., as well known in the art.

“Treatment,” “treating,” and “treat” are defined as acting upon a disease, disorder, or condition with an agent to reduce or ameliorate harmful or any other undesired effects of the disease, disorder, or condition and/or its symptoms. “Treatment,” as used herein, covers the treatment of a subject in need thereof, and includes treatment of cardiovascular disease, for example, heart failure, myocardial ischemia, hypoxia, stroke, myocardial infarction and chronic ischemic heart disease. “Treating” or “treatment of” a condition or subject in need thereof refers to (1) taking steps to obtain beneficial or desired results, including clinical results such as the reduction of symptoms; (2) preventing the disease, for example, causing the clinical symptoms of the disease not to develop in a patient that may be predisposed to the disease but does not yet experience or display symptoms of the disease; (3) inhibiting the disease, for example, arresting or reducing the development of the disease or its clinical symptoms; (4) relieving the disease, for example, causing regression of the disease or its clinical symptoms; or (5) delaying the disease. For purposes of this invention, beneficial or desired clinical results include, but are not limited to, generating an induced cardiomyocyte and/or promoting myocardial regeneration.

The term “WNT” is meant to include a family of highly conserved secreted signaling molecules which play key roles in both embryogenesis and mature tissues. The human WNT gene family has at least 19 members (WNT-1, WNT-2, WNT-2B/WNT-13, WNT-3, WNT3a, WNT-4, WNT-5A, WNT-5B, WNT-6, WNT-7A, WNT-7B, WNT-8A, WNT-8B, WNT-9A/WNT-14, WNT-9B/WNT-15, WNT-10A, WNT-10B, WNT-11, WNT-16). WNT proteins modulate cell activity by binding to WNT receptor complexes that include a polypeptide from the Frizzled (Fz) family of proteins and a polypeptide of the low-density lipoprotein receptor (LDLR)-related protein (LRP) family of proteins. Once activated by WNT binding, the WNT receptor complex will activate one or more intracellular signaling cascades. These include the canonical WNT signaling pathway; the WNT/planar cell polarity (WNT/PCP) pathway; and the WNT-calcium (WNT/Ca2+) pathway.

### Reprogramming Methods

Non-cardiomyocytes cells can be differentiated into cardiomyocytes cells in vitro or in vivo using any method available to one of skill in the art. For example, see methods described in Ieda et al. (2010) Cell 142:375-386; Christoforou et al. (2013) PLoS ONE 8:e63577; Addis et al. (2013) J. Mol. Cell Cardiol. 60:97-106; Jayawardena et al. (2012) Circ. Res. 110:1465-1473; Nam Y et al., PNAS USA. 2013; 110:5588-5593; Wada R et al. PNAS USA. 2013; 110:12667-12672; and Fu J et al., Stem Cell Reports. 2013; 1:235-247.

In some embodiments, the reprogramming factors used are selected from the group consisting of Baf60c, Esrrg, Gata4, Gata6, Hand2, Irx4, Isl1, Mef2c, Mesp1, Mesp2, Myocardin, Nkx2.5, SRF, Tbx5, Tbx20, Zfpm2, miR-133, or any combination thereof. In some embodiments, one or more of the above listed reprogramming factors is expressly excluded.

In some embodiments, the reprogramming factors used are Gata4, Mef2c, and Tbx5 (i.e., GMT). In some embodiments, the reprogramming factors are Myocardin, Mef2c, and Tbx5 (i.e., MMT). In some embodiments, the reprogramming factors are Gata4, Mef2c, Tbx5, and Myocardin (i.e., 4F). In other embodiments, the reprogramming factors are Gata4, Mef2c, and Tbx5, Essrg, Myocardin, Zfpm2, and Mesp1 (i.e., 7F).

The reprogramming factors can be introduced to the non-cardiomyocyte by a variety of mechanisms commonly known to those of skill in the art. For example, viral constructs can be delivered through the production of a virus in a suitable host. Virus is then harvested from the host cell and contacted with the cardiac cell. Viral and non-viral vectors capable of expressing genes of interest can be delivered to a non-cardiomyocyte via DNA/liposome complexes, micelles and targeted viral protein-DNA complexes.

Liposomes that also comprise a targeting antibody or fragment thereof can be used in the methods of this invention. In addition to the delivery of polynucleotides to a non-cardiomyocyte or cell population, direct introduction of proteins described herein to the non-cardiomyocyte or cell population can be done by the non-limiting technique of protein transfection, alternatively culturing conditions that can enhance expression and/or promote activity of the proteins of this invention are other non-limiting techniques.

Other methods of delivering vectors encoding reprogramming factors include, but are not limited to, calcium phosphate transfection, DEAE-dextran transfection, electroporation, microinjection, protoplast fusion, or liposome-mediated transfection. The host cells that are transfected with the vectors of this invention may include, but are not limited to, E. coli or other bacteria, yeast, fungi, or cells derived from mice, humans, or other animals (e.g., mammals). In vitro expression of a protein, fusion protein, polypeptide fragment or mutant encoded by cloned DNA may also be used. Those skilled in the art of molecular biology will understand that a wide variety of expression systems and purification systems may be used to produce recombinant proteins and fragments thereof.

In some embodiments, the non-cardiomyocyte is first contacted with a TGF-β inhibitor at between about 12 hours and about 36 hours after the reprogramming factor has been introduced. In one preferred embodiment, the non-cardiomyocyte is first contacted with a TGF-β inhibitor at about 24 hours after the reprogramming factor has been introduced.

In some embodiments, the non-cardiomyocyte is first contacted with a WNT inhibitor at between about 24 hours and about 72 hours after the reprogramming factor has been introduced. In one preferred embodiment, the non-cardiomyocyte is first contacted with the WNT inhibitor at about 48 hours after the reprogramming factor has been introduced.

### Cells and Culture Conditions

As will be apparent to the skilled artisan upon reading this disclosure, the present disclosure provides methods for generating induced cardiomyocytes and cardiomyocyte-like cells from non-cardiomyocytes.

### Cells

The non-cardiomyocyte for use in the present invention can be any non-cardiomyocyte known to one of skill in the art. Non-limiting examples of a non-cardiomyocyte include, for example, a somatic cell, a cardiac fibroblast, a non-cardiac fibroblast, a cardiac progenitor cell, and a stem cell. The non-cardiomyocyte can be cardiac cells from the epicardium, myocardium or endocardium of the heart. Non-cardiomyocyte cardiac cells include, for example, smooth muscle and endothelial cells. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure.

In some embodiments the non-cardiomyocytes are endogenous cells within the subject and the methods of generating induced cardiomyocytes are by in vivo induction. In other embodiments, the non-cardiomyocytes are exogenous and are modified in vitro.

The non-cardiomyocytes that are induced to cardiomyocytes can be from any of a variety of sources. Mammalian non-cardiomyocytes (e.g., human or murine) can be used. In some embodiments, the cardiomyocytes are mammalian cardiomyocytes, and in specific embodiments the non-cardiomyocytes are human cells. In some embodiments, the non-cardiomyocytes can be derived from stem cells (e.g., pluripotent stem cells, induced pluripotent stem cells, reprogrammed cardiac cells or cardiac stem cells) or progenitor cells (e.g., cardiac progenitor cells). Cardiomyocytes can be derived from cardiac or non-cardiac cells. Cardiomyocytes can be from or derived from any of a variety of tissue sources. For example, cardiac fibroblasts, foreskin fibroblast, dermal fibroblasts, lung fibroblasts, etc. The non-cardiomyocytes can be embryonic, fetal, or post-natal (e.g., adult) cells. In preferred embodiments, the non-cardiomyocytes are adult cells.

The non-cardiomyocytes can be obtained from a living subject. The cells can be obtained from tissue taken from a living subject. The cells can be obtained from a recently deceased subject who is considered a suitable tissue donor. In some embodiments, the subject is screened for various genetic disorders, viral infections, etc. to determine whether the subject is a suitable source of cells. In general, a cell that is suitable for use in the present invention is non-transformed (e.g., exhibits normal cell proliferation) and is otherwise normal (e.g., exhibits normal karyotype).

Where the cells for reprogramming are a population of non-cardiomyocytes, the population of cells is composed of at least about 30% non-cardiomyocytes, at least about 35% non-cardiomyocytes, at least about 40% non-cardiomyocytes, at least about 45% non-cardiomyocytes, at least about 50% non-cardiomyocytes, at least about 55% non-cardiomyocytes, at least about 60% non-cardiomyocytes, at least about 65% non-cardiomyocytes, at least about 70% non-cardiomyocytes, at least about 75% non-cardiomyocytes, at least about 80% non-cardiomyocytes, at least about 85% non-cardiomyocytes, at least about 90% non-cardiomyocytes, at least about 95% non-cardiomyocytes, at least about 98% non-cardiomyocytes, at least about 99% non-cardiomyocytes, or greater than 99% non-cardiomyocytes.

Cells (cardiac or non-cardiac) can be derived from tissue of a non-embryonic subject, a neonatal infant, a child or an adult. Cells can be derived from neonatal or post-natal tissue collected from a subject within the period from birth, including cesarean birth, to death. For example, the non-cardiomyocytes can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

Methods of isolating non-cardiomyocytes cells from tissues are known in the art, and any known method can be used. As a non-limiting example, adult cardiac cells can be obtained from human heart atrial biopsy specimens obtained from patients undergoing cardiac surgery. Cardiac tissue can be minced and digested with collagenase and cardiac stem/progenitor cells expanded in c-kit+ progenitor cell expansion media using the methods of Choi et al. (2013) Transplantation Proceedings 45:420-426. In addition, cardiac fibroblasts can be obtained using the methods of Ieda et al. (2009) Dev. Cell 16(2):233-244. Foreskin fibroblasts can be obtained from foreskin tissue of a male individual. The fibroblasts can be obtained by mincing the foreskin tissue, then dissociating the tissue to single cells. Foreskin cell clumps can be dissociated by any means known in the art including physical de-clumping or enzymatic digestion using, for example, trypsin.

The expression of various markers specific to cardiomyocytes may be detected by conventional biochemical or immunochemical methods (e.g., enzyme-linked immunosorbent assay, immunohistochemical assay, and the like). Alternatively, expression of a nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting and analyzing mRNA coding for any marker proteins. Nucleic acid sequences coding for markers specific to cardiomyocytes are known and are available through public databases such as GenBank. Thus, marker-specific sequences needed for use as primers or probes are easily determined

### Culture Conditions

The cells of the present disclosure can be cultured under any conditions known to one of skill in the art. In some embodiments, the cells (e.g., non-cardiomyocytes, cardiomyocytes, and combinations thereof) are cultured in conditions of 1-20% oxygen (O₂) and 5% carbon dioxide (CO₂). In some embodiments, the cells of the present disclosure are cultured under hypoxic conditions (e.g., in the presence of less than 10% O₂). In some embodiments, the cells of the present disclosure are cultured at about 37° C. In some embodiments, the cells of the present disclosure can be cultured at about 37° C., 5% CO₂ and 10-20% O₂. In some embodiments, the cells are cultured in hypoxic conditions for a period of time. For example, the cells may be cultured under normoxic conditions (˜20% O₂) for a period of time and then switched to hypoxic conditions, for example ˜5% O₂.

The advantage of in vitro or ex vivo differentiating of non-cardiomyocytes to cardiomyocytes is the ability to easily identify cells suitable for implantation or for discrimination of cells that are damaged or have not differentiated. In vitro or ex vivo differentiation allows induced cardiomyocytes to be purified or isolated from non-cardiomyocytes that have not differentiated.

In some embodiments, a non-cardiomyocyte is induced using an effective amount of an agent (a small molecule or siRNA) to generate an induced cardiomyocyte or a cardiomyocyte-like cell.

In some embodiments, the agent is a small molecule selected from the group consisting of SB431542, LDN-193189, dexamethasone, LY364947, D4476, myricetin, IWR1, XAV939, docosahexaenoic acid (DHA), S-Nitroso-N-acetylpenicillamine (SNAP), Hh-Ag1.5, alprostadil, cromakalim, MNITMT, A769662, retinoic acid p-hydoxyanlide, decamethonium dibromide, nifedipine, piroxicam, bacitracin, aztreonam, harmalol hydrochloride, amide-C2 (A7), Ph-C12 (C10), mCF3-C-7 (J5), G856-7272 (A473), 5475707, or any combination thereof.

### Agents for Reprogramming

### Transforming Growth Factor Beta (TGF-β) Inhibitors

Transforming growth factor-beta (TGF-β) is a multifunctional regulatory polypeptide that is the prototypical member of a large family of cytokines that controls many aspects of cellular function. The TGF-β pathway influences fibrosis, apoptosis, transdifferentiation, proliferation, and other cellular function. Leask, A. (2015) Circ. Res. 116:1269-1276; Xu, J., et al. (2009) Cell Res. 19:156-172; Schmierer, B. et al (2007) Nat. Rev. Mol. Cell. Biol. 8:970-982. TGFβ signaling deregulation is frequent in tumors and has crucial roles in tumor initiation, development and metastasis. TGFβ signaling inhibition is an emerging strategy for cancer therapy.

A TGF-β inhibitor is a compound that inhibits TGF-β signal transduction by inhibiting any of the factors constituting the TGF-β signal transduction system pathway, such as TGF-β ligand, TGF-β Type I receptors, TGF-β Type II receptors, TGF-β Type III receptors (β-glycan and endoglin), soluble forms of the TGF-β receptors, Smad proteins, antibodies against receptors and ligands implicated in the signaling pathway, nucleic acid based molecules (e.g., antisense, siRNA, aptamers and ribozymes) targeting the pathway members, or a combination thereof.

In some embodiments, the TGF-β inhibitor is selected from the group consisting of SB431542, D4476, LDN-193189, dexamethasone and LY364947. TGF-β inhibitors also may be referred to in the art as anti-TGF-β compounds. Non-limiting examples of anti-TGF-β compounds include, antibodies (e.g., Fresolumimab/GC1008 (Genzyme, Cambridge, Mass., USA), PF-03446962 (Pfizer, New York, N.Y., USA)), antisense oligonucleotides (ASO) (e.g., Trabedersen (AP12009) (Isarna Therapeutics, New York, N.Y., USA)), receptor kinase inhibitors (e.g., LY2157299 (Eli Lilly, Indianapolis, Ind., USA), and combined TGF-β ASO with a vaccine (e.g., Lucanix™ (Belagenpumatucel-L) (Nova Rx Corp, San Diego, Calif., USA), and TGF-β2 ASO+GMCSF expression vector (Mary Crowley Medical Research Centre, Dallas, Tex., USA)).

### WNT Inhibitors

WNT inhibitors are agents that downregulate expression or activity of WNT. WNT/β-catenin signaling is involved in abroad range of biological systems including stem cells, embryonic development and adult organs. Deregulation of components involved in WNT/β-catenin signaling has been implicated in a wide spectrum of diseases including a number of cancers and degenerative diseases. WNT signaling occurs through three major pathways: canonical, noncanonical planar-cell polarity, and noncanonical WNT/calcium. In the canonical pathway, WNT binds frizzled to disrupt the function of a complex that targets β-catenin for ubiquitination and degradation in the proteasome. The roles of WNT signaling in stem-cell renewal, induced pluripotent stem (iPS) cell reprogramming, and cell differentiation to various lineages are still debated.

However, biphasic modulation of WNT signaling during stem-cell differentiation into cardiomyocytes promotes mesoderm differentiation and produces a high yield of pure cardiomyocytes. Agents of interest may interact directly with WNT, e.g. drugs, i.e., small molecules, blocking antibodies, etc., or may interact with WNT associated proteins, e.g. WNT co-receptors LRP5/6 and the transmembrane protein Kremen. A number of WNT inhibitors have been described and are known in the art.

WNT inhibitors of interest interfere with the interaction between soluble, extracellular WNT proteins, and the frizzled receptors that are present on the surface of normal cells. Such agents include, without limitation, soluble frizzled polypeptides comprising the WNT binding domains; soluble frizzled related polypeptides; WNT specific antibodies; frizzled specific antibodies; and other molecules capable of blocking extracellular WNT signaling.

Among the known WNT inhibitors are members of the Dickkopf (Dkk) gene family (see Krupnik et al. (1999) Gene 238(2):301-13). Members of the human Dkk gene family include Dkk-1, Dkk-2, Dkk-3, and Dkk-4, and the Dkk-3 related protein Soggy (Sgy). Other inhibitors of WNT include Wise (Itasaki et al. (2003) Development 130(18):4295-30), which is a secreted protein. The Wise protein physically interacts with the WNT co-receptor, lipoprotein receptor-related protein 6 (LRP6), and is able to compete with WNT8 for binding to LRP6.

Inhibitors may also include derivatives, variants, and biologically active fragments of native inhibitors.

In some embodiments, the WNT inhibitor is a small molecule such as XAV939, D4476, IWR1, IWR analogs, IWP analogs, 53AH, WNT-059 and myricetin. Non-limiting examples of WNT-targeting compounds include, OMPT-18RS (OncoMed Pharmaceuticals/Bayer, Redwood City, Calif., USA), OMP-54F28 (OncoMed Pharmaceuticals/Bayer, Redwood City, Calif., USA), PRI-724 (Prism Pharma Co, Ltd/Eisai, Yokohama, JP), LGK974 (Novartis Pharmaceuticals, East Hanover, N.J., USA), and JW55 (Tocris Bioscience, Bristol, UK).

In specific embodiments, the WNT inhibitor is an siRNA that targets WNT activity. Examples of siRNA WNT inhibitors include siRNAs that interfere with the expression of WNT itself, or siRNAs that interfere with the expression of a molecules necessary for transduction in the WNT signaling pathway, e.g., Tankyrase 1

### Anti-Inflammatory Agents

In certain embodiments, the reprogramming is enhanced by the administration of one or more anti-inflammatory agents, e.g., an anti-inflammatory steroid or a nonsteroidal anti-inflammatory drug (NSAID).

Anti-inflammatory steroids for use in the invention include the corticosteroids, and in particular those with glucocorticoid activity, e.g., dexamethasone and prednisone. Non-steroidal anti-inflammatory drugs (NSAIDs) for use in the invention generally act by blocking the production of prostaglandins that cause inflammation and pain, cyclooxygenase-1 (COX-1) and/or cyclooxygenase-2 (COX-2). Traditional NSAIDs work by blocking both COX-1 and COX-2. The COX-2 selective inhibitors block only the COX-2 enzyme. In certain embodiment, the NSAID is a COX-2 selective inhibitor, e.g., celecoxib (Celebrex®), rofecoxib (Vioxx®), and valdecoxib (Bextra®). In certain embodiments, the anti-inflammatory is an NSAID prostaglandin inhibitor, e.g., Piroxicam.

The dosage of the anti-inflammatory agent for use in the invention can be determined based on knowledge of the particular anti-inflammatory agent used, the other treatments and medications a subject may be receiving, and the other needs of the subject(s) as will be known by those of ordinary skill in the art. For example, celecoxib is conventionally prescribed in 100 mg or 200 mg capsules and the general dose (e.g., for pain caused by arthritis) is generally between 100-400 mg a day.

### Methods for In Vitro Modification

The disclosure provides methods for generating cardiomyocytes and/or cardiomyocyte-like cells in vitro.

In some aspects, provided herein are methods for generating an induced cardiomyocyte, the method comprising: culturing a non-cardiomyocyte in the presence of an effective amount of a WNT inhibitor, thereby generating an induced cardiomyocyte or a cardiomyocyte-like cell.

In some aspects, provided herein are methods for generating an induced cardiomyocyte, the method comprising: a) administering to a non-cardiomyocyte an effective amount of a TGF-β inhibitor for a first period of time prior to the addition of a WNT inhibitor; and b) administering to the non-cardiomyocyte an effective amount of the TGF-β inhibitor concurrently with an effective amount of the WNT inhibitor for a second period of time.

In some embodiments, the methods comprise culturing the non-cardiomyocyte in the presence of an effective amount of a TGF-β inhibitor.

In some embodiments, the non-cardiomyocyte is cultured for a period of time in the presence of the TGF-β inhibitor prior to the addition of the WNT inhibitor, for example, between about 6 hours and about 72 hours prior to addition of the WNT inhibitor. In some embodiments, the non-cardiomyocyte is cultured for between about 12 hours and about 60 hours, about 18 hours and about 48 hours, about 24 hours and about 42, about 30 hours and about 36 hours in the presence of the TGF-β inhibitor prior to the addition of the WNT inhibitor (or any ranges between any two of the numbers, end points inclusive). In some embodiments the non-cardiomyocyte is cultured for about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, or about 72 hours in the presence of the TGF-β inhibitor prior to the addition of the WNT inhibitor.

In some embodiments, the non-cardiomyocyte is cultured in the presence of the TGF-β inhibitor for about 6 hours, about 12 hours, about 18 hours, about 24 hours, about 30 hours, about 36 hours, about 42 hours, or about 48 hours prior to addition of the WNT inhibitor. In one preferred embodiment, the non-cardiomyocyte is cultured in the presence of the TGF-β inhibitor for about 24 hours prior to addition of the WNT inhibitor.

In some embodiments, the non-cardiomyocyte is cultured in the presence of the TGF-β inhibitor and the WNT inhibitor concurrently for a period of time.

In another embodiment, the non-cardiomyocyte is cultured in the presence of a TGF-β activator subsequent to the TGF-β inhibitor. TGF-β activators are well-known in the art. A non-limiting example of a commercially available TGF-β activator includes, for example, SD 208 (Tocris Bioscience, Bristol, UK).

In some embodiments, the WNT inhibitor, the TGF-β inhibitor, or both are removed after day 1, day 2, day 3, day 4, day 5, day 6, day 7, day 8, day 9, day 10, day 11, day 12, day 13, day 14, day 15, day 20, day 25, day 30, 1 month, 2 months, 3 months, or 4 months of reprogramming. In some embodiments, the WNT inhibitor, the TGF-β inhibitor, or both are removed after day 10 of reprogramming.

### Compositions

The present disclosure also provides isolated induced cardiomyocytes generated according to the methods of the invention. The induced cardiomyocytes may express at least one cardiac gene at a level higher or a level lower than that found in a naturally occurring cardiomyocyte.

In some embodiments, the cardiac gene expressed at a higher level than that found in the naturally occurring cardiomyocyte is selected from the group consisting of Tnnt2, Actn2, Atp2a2, Myh6, Ryr2, Myh7, and Actcl. In some embodiments, the cardiac gene expressed at a lower level than that found in the naturally occurring cardiomyocyte is selected from the group consisting of Mybpc3, Pln, Mb, Lmod2, My12, My13, Cox6a2, Atp5a1, Ttn, Tnni3, Pdk4, Mycz2, Cacna1c, Scn5a, Mycod, and Nppa.

In another aspect, a substantially homogenous population of induced cardiomyocytes generated according to the methods of the invention are provided. In some embodiments, the induced cardiomyocytes of the substantially homogenous population express at least one cardiac gene at a higher level or a lower level that found in a naturally occurring cardiomyocyte.

In some embodiments, the composition comprises a population of isolated induced cardiomyocytes described herein and a carrier, optionally a pharmaceutically acceptable excipient. In some embodiments, the compositions further comprise a stabilizer and/or a preservative.

The composition may comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like.

Parenteral compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

The composition can include agents that are administered using an implantable device. Suitable implantable devices contemplated by this disclosure include intravascular stents (e.g., self-expandable stents, balloon-expandable stents, and stent-grafts), scaffolds, grafts, and the like. Such implantable devices can be coated on at least one surface, or impregnated, with a composition capable of generating an induced cardiomyocyte. The composition can also include agents that are contained within a reservoir in the implantable device. Where the agents are contained within a reservoir in the implantable device, the reservoir is structured so as to allow the agents to elute from the device. The agents of the composition administered from the implantable device may comprise a WNT inhibitor, the TGF-β inhibitor or both.

Pharmaceutical compositions can be provided in any form amenable to administration. Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

To control tonicity, an aqueous pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition is preferably sterile. The composition is preferably gluten free. The composition is preferably non-pyrogenic.

The pharmaceutical composition can be administered by any appropriate route, which will be apparent to the skilled person depending on the disease or condition to be treated. Typical routes of administration include oral, intravenous, intra-arterial, intramuscular, subcutaneous, intracranial, intranasal or intraperitoneal.

In some embodiments, a composition comprising cells may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In some embodiments, one or more agents used in the methods of the invention is provided in a controlled release formulation. The term “controlled release formulation” includes sustained release and time-release formulations. Controlled release formulations are well-known in the art. These include excipients that allow for sustained, periodic, pulse, or delayed release of the composition. Controlled release formulations include, without limitation, embedding of the composition (a WNT inhibitor and/or TGF-β inhibitor) into a matrix; enteric coatings; micro-encapsulation; gels and hydrogels; implants; and any other formulation that allows for controlled release of a composition.

In one aspect is provided a kit of parts comprising the above-mentioned agents, compositions or formulations.

### Methods of Treatment

Subjects in need of treatment using the compositions, cells and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Subjects who are suitable for treatment using the compositions, cells and methods of the present disclosure include individuals (e.g., mammalian subjects, such as humans, non-human primates, domestic mammals, experimental non-human mammalian subjects such as mice, rats, etc.) having a cardiac condition including but limited to a condition that results in ischemic heart tissue (e.g., individuals with coronary artery disease) and the like. In some examples, an individual suitable for treatment suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

### In Vivo Generation of Induced Cardiomyocytes

In some aspects are provided methods of treating a cardiovascular disease comprising administering to a subject in need thereof an effective amount of a WNT inhibitor, a TGF-β inhibitor, and one or more reprogramming factors, thereby generating an induced cardiomyocyte or a cardiomyocyte-like cell. In some embodiments, the TGF-β inhibitor and the WNT inhibitor are administered concurrently with the reprogramming factors. In other embodiments, the TGF-β inhibitor and the WNT inhibitor are administered concurrently with one another. In yet other embodiments, the TGF-β inhibitor and the WNT inhibitor are administered sequentially with each other and/or the reprogramming factors.

In some embodiments, the TGF-β inhibitor is administered for a period of time prior to the WNT inhibitor, for example, between about 6 hours and about 72 hours prior to administration of the WNT inhibitor. In some embodiments, the TGF-β inhibitor is delivered between about 12 hours and about 60 hours, about 18 hours and about 48 hours, about 24 hours and about 42, about 30 hours and about 36 hours in the presence of the TGF-β inhibitor prior to the addition of the WNT inhibitor (or any ranges between any two of the numbers, end points inclusive). In some embodiments, the TGF-β inhibitor is delivered to the subject about 1 hour, about 2 hours, about 3 hours, about 4 hours, about 5 hours, about 6 hours, about 12 hours, about 18 hours, about 24 hours, about 30 hours, about 36 hours, about 42 hours, about 48 hours, about 60 hours, or about 72 hours prior to the addition of the WNT inhibitor.

The WNT inhibitor, the TGF-β inhibitor or both can be administered by any method known by one of skill in the art. For example, the WNT inhibitor, the TGF-β inhibitor or both can be achieved by, for example, oral delivery, intravascular delivery, intramuscular delivery, intramyocardial delivery, intraperitoneally delivery, and the like.

The WNT inhibitor, the TGF-β inhibitor or both can be administered following any schedule, for example, every day, every other day, twice a day, every two, every three, every four, every five days, and so on. In some embodiments, the WNT inhibitor, the TGF-β inhibitor or both are administered every day for approximately 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, or more. In some embodiments, the WNT inhibitor, the TGF-β inhibitor or both are administered every day for approximately 14 days.

In some embodiments, the subject is administered the TGF-β inhibitor and the WNT inhibitor concurrently for a period of time.

In some embodiments, the subject is administered a TGF-β activator subsequent to the TGF-β inhibitor. TGF-β activators are well-known in the art. A non-limiting example of a commercially available TGF-β activator includes, for example, SD 208 (Tocris Bioscience, Bristol, UK).

In some embodiments, at least one reprogramming factor has been administered to the subject, for example, Baf60c, Esrrg, Gata4, Gata6, Hand2, Irx4, Isl1, Mef2c, Mesp1, Mesp2, Myocardin, Nkx2.5, SRF, Tbx5, Tbx20, Zfpm2, miR-133, or any combination thereof. In preferred embodiments, a combination of two or more, and more preferably three or more, of the reprogramming factors are administered to the subject. In some embodiments, one or more of the above listed reprogramming factors is expressly excluded. In other embodiments, the reprogramming factors are selected from the group of Gata4, Mef2c, Tbx5, Myocardin, or any combination thereof. In specific embodiments, the reprogramming factors are Gata4, Mef2c, and Tbx5 (GMT). In other specific embodiments, the reprogramming factors are Myocardin, Mef2c, and Tbx5 (i.e., MMT). In other specific embodiments, the reprogramming factors are Gata4, Mef2c, Tbx5, and Myocardin (i.e., 4F). In other embodiments, the reprogramming factors are Gata4, Mef2c, and Tbx5, Essrg, Myocardin, Zfpm2, and Mesp1 (i.e., 7F).

In some embodiments, a TGF-β inhibitor is administered to the subject at between about 12 hours and about 36 hours after the reprogramming factor has been introduced. Non-limiting examples of TGF-β inhibitors include SB431542, D4476, LDN-193189, dexamethasone and LY364947. siRNA targeting TGF-β, a TGF-β receptor, or a member of the TGF-β signaling pathway can also be used with the present invention.

In some embodiments, a WNT inhibitor is administered to the subject at between about 24 hours and about 72 hours after the reprogramming factor has been introduced. In some embodiments, the WNT inhibitor is selected from the group consisting of XAV939, D4476, IWR1, and myricetin.

### Methods of Treatment Using Induced Cardiomyocytes

The invention provides methods of treating a cardiovascular disease comprising administering to a subject in need thereof an effective amount of an induced cardiomyocyte produced by the methods described herein.

In some aspects, an induced cardiomyocyte of the present disclosure can be used to treat a subject in need thereof. In some embodiments, the induced cardiomyocyte can be administered to the subject in need thereof, where administration into the subject of the induced cardiomyocyte, treats a cardiovascular disease in the subject. Thus, in some embodiments, a method of treating cardiovascular disease involves administering to a subject in need thereof a population of induced cardiomyocyte. In other embodiments, a method of treating cardiovascular disease involves administering to the subject in need thereof an effective amount of a composition comprising a WNT inhibitor, the TGF-β inhibitor or both.

In some embodiments, the non-cardiomyocyte is cultured in the presence of the TGF-β inhibitor for about 6 hours, about 12 hours, about 18 hours, about 24 hours, about 30 hours, about 36 hours, about 42 hours, or about 48 hours prior to addition of the WNT inhibitor. In one preferred embodiment, the non-cardiomyocyte is cultured in the presence of the TGF-β inhibitor for about 24 hours prior to addition of the WNT inhibitor.

Unless stated otherwise, the abbreviations used throughout the specification have the following meanings: α-MHC-GFP, alpha-myosin heavy chain green fluorescence protein; CF, cardiac fibroblast; cm, centimeter; CO, cardiac output; EF, ejection fraction; FACS, fluorescence activated cell sorting; GFP, green fluorescence protein; GMT, Gata4, Mef2c and Tbx5; GMTc, Gata4, Mef2c, Tbx5, TGFβi, WNTi; GO, gene ontology; HCF, human cardiac fibroblast; iCM, induced cardiomyocyte; kg, killigram; microgram; μl, microliter; mg, milligram; ml, milliliter; MI, myocardial infarction; msec, millisecond; min, minute; MMT, Myocardin, Mef2c and Tbx5; MMTc, Myocardin, Mef2c, Tbx5, TGFβi, WNTi; MRI, magnetic resonance imaging; PBS, phosphate buffered saline; PBST, phosphate buffered saline, triton; PFA, paraformaldehyde; qPCR, quantitative polymerase chain reaction; qRT-PCR, quantitative reverse transcriptase polymerase chain reaction; RNA, ribonucleic acid; RNA-seq, RNA sequencing; RT-PCR, reverse transcriptase polymerase chain reaction; sec, second; SV, stroke volume; TGF-β, transforming growth factor beta; TGF-βi, transforming growth factor beta inhibitor; WNT, wingless-Int; WNTi, wingless-Int inhibitor; YFP, yellow fluorescence protein; 4F, Gata4, Mef2c, TBX5, and Myocardin; 4Fc, Gata4, Mef2c, TBX5, and Myocardin+TGF-βi and WNTi; 7F, Gata4, Mef2c, and Tbx5, Essrg, Myocardin, Zfpm2, and Mesp1; 7Fc, Gata4, Mef2c, and Tbx5, Essrg, Myocardin, Zfpm2, and Mesp1+TGF-βi and WNTi.

### EXAMPLES

The following examples are intended to further illustrate certain embodiments of the disclosure. The examples are put forth so as to provide one of ordinary skill in the art and are not intended to limit its scope.

### Example 1. TGF-β and WNT Signaling are Barriers to Cardiac Reprogramming

To identify biological pathways that could be manipulated to improve cardiac reprogramming, a chemical biology approach involving an unbiased small molecule library screen was used. Cardiac reprogramming was induced with the recently described polycistronic GMT retrovirus Mef2c-P2A-Gata4-T2A-Tbx5 to ensure that each of the three transcription factors are optimally expressed, as described in detail in Wang et al. (2015) Circ. Res. 116:237-244.

Briefly, mouse cardiac fibroblasts (CF) were isolated from PO-P4 αMHC-GFP transgenic neonates using the migration method as previously described. Burridge et al. (2012) Cell Stem Cell 10:16-28; Ieda et al. (2010) Cell 142:375-386. Hearts were isolated, minced, and onto gelatin-coated plates and incubated in fibroblast explant media (20% FBS in IMOM) for one week at 37° C. The tissue was washed with 2×PBS, digested in 0.05% Trypsin for 5 minutes, and resuspended in fibroblast explant media. Tissues were filtered through a 70 μM filter and pelleted. Pelleted cells were stained for 20 minutes with Thy-1-APC (anti-mouse/rat CD90.1 thy-1.1) (Ebioscience, San Diego, Calif., USA) and washed 2× with PBS as previously described. Ieda et al. (2010) Cell 142:375-386. Cells were sorted for APC⁺ cells by fluorescence activated cell sorting (FACS) and plated onto 10 cm gelatin-coated plates and used fresh for all studies without freezing.

Direct conversion of Thy1⁺ CFs to iCMs was completed as previously described in Ieda et al. (2010) Cell 142:375-386. Briefly, pMXs-Gata4, pMXs-Mef2c, pMXs-Tbx5, polycistronic pMXs-Mef2c-Gata4-Tbx5 (GMT polycistronic) or pMXs-dsRed were constructed as previously described. Ieda et al. (2010) Cell 142:375-386; Wang, L., et al. (2015) Circ Res 116, 237-244 (2015). Retroviral vectors were packaged using Fugene HD (Roche) and delivered in OptiMEM (10 μg) to 15-cm plates containing ˜80% confluent PlatE cells in fibroblast explant media, as previously described. Qian, L., et al. (2012) Nature 485, 593-59. Viral supernatant was collected 48 hours post-transfection and used to infect cardiac fibroblasts with the addition of 0.6 μg/ml polybrene (Chemicon) and added to cardiac fibroblasts at day −1. After 24 hours, the culture medium was replaced with cardiomyocyte culture medium (iCM medium (Qian, L., et al. (2013) Nature Protocols 8: 1204-1215)) at day 0, and replaced every 3-4 days. Three separate Gata4, Mef2c, and Tbx5 retroviruses were used in the initial drug screening and the in vivo experiments; however for further in vitro experiments following the initial screening the GMT polycistronic retrovirus was used.

For drug screening, Thy1⁺ neonatal cardiac fibroblasts from α-MHC-GFP transgenic mice were plated in 384-well plates at a density of 2000 cells/well. Cells were reprogrammed with the GMT retrovirus as described above. At day 1 of reprogramming, the virus was replaced with iCM medium and the compounds were added to the wells using a Biomek liquid-handling robotic station to achieve a 1:1000 dilution of the drug-library concentration. Libraries of 5500 toxicologically tested compounds (e.g., LOPAC, TOCRIS, LONZA, and SPECTRUM) were screened. At day 14 of reprogramming, the plates were fixed and stained for GFP and Troponin T as described under immunocytochemistry and imaged using high-throughput, high-content imaging with an INCELL system. Data were analyzed using the INCELL image-analysis package.

For immunocytochemical analysis and quantification, samples were fixed using 4% paraformaldehyde (PFA), washed twice in PBS containing 0.1% Triton X-100 (PBST), and blocked with Power Block Universal Blocking Reagent (Biogenex, # HK085-5K) for 15 minutes at room temperature as previously described. Qian et al. (2013) Nature protocols 8:1204-1215. Samples were incubated in a mixture containing primary antibodies diluted in PBST and blocking buffer (1:1) and incubated at 4° C. overnight. The following primary antibodies were used: troponin T, cardiac isoform Ab-1, mouse monoclonal antibody (Thermo Scientific # MS-295-PO, 1:200), monoclonal anti-α-actinin sarcomeric antibody (Sigma Aldrich # A7811, 1/200), and anti-GFP (Thermo Scientific # A11120, 1:200). Samples were washed twice with PBST for 15 minutes each at room temperature and then incubated in a mixture containing secondary antibodies diluted in PBST and blocking buffer (1:1) at room temperature for 1 hour. Samples were washed three times with PBST for 15 minutes each at room temperature and visualized. Stained cells were quantified with ImageJ Imaging Analysis Software. The standard error mean was calculated for all comparisons; p<0.05 was considered statistically significant.

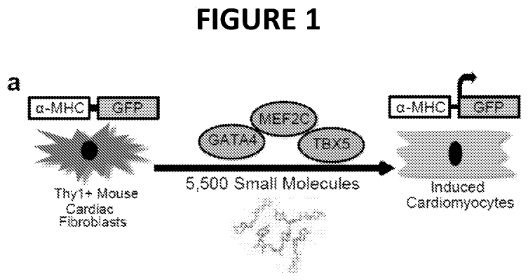

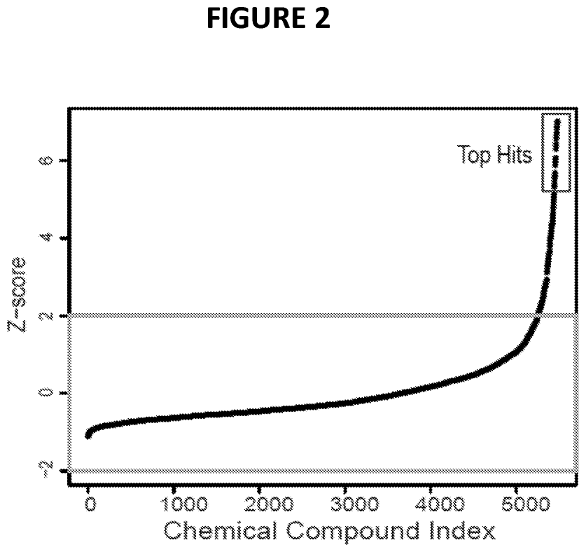

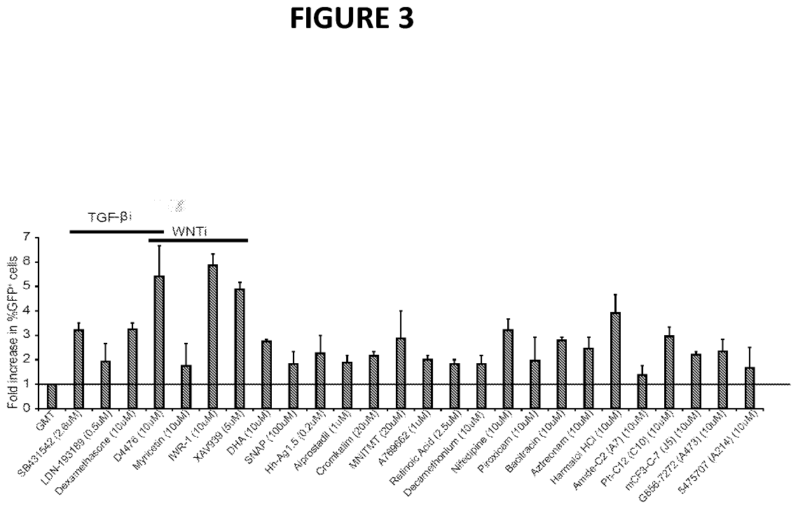

Potential iCMs were detected by activation of the α-MHC-driven GFP reporter. After optimizing this method of direct reprogramming in a 384-well format, the compounds from libraries of toxicologically tested compounds (e.g., LOPAC, TOCRIS, and SPECTRUM drug libraries) were screened using high-throughput, high-content imaging (FIG. 1). Compounds were added one day after GMT transduction, and α-MHC-GFP⁺ cells were quantified after 2 weeks. Twenty-six top hits with a Z score over 5 (FIG. 2) were identified and following validation, these hits increased the percentage of GFP iCMs by two- to six-fold (FIG. 3). The top hits included three molecules that inhibit TGF-β signaling (SB431542, LDN-193189, and dexamethasone), three that inhibit WNT signaling (XAV939, IWR1, and myricetin), and one that inhibits both WNT and TGF-β signaling (D4476), as well as molecules with anti-inflammatory properties (e.g., dexamethasone, DHA and piroxicam (FIG. 3). The multiple hits on common pathways suggested these signaling pathways were impacting cardiac reprogramming.

To identify which of the WNT or TGF-β inhibitor compounds most effectively improve reprogramming quality, qRT-PCR was performed for a panel of cardiac and fibroblast genes. Briefly, total RNA was isolated with a commercial kit (Direct-Zol RNA Mini-prep, Zymo Research). Reverse transcription was carried out using a mixture of oligo(dT) and random hexamer primers (SuperScript III First-Strand Synthesis SuperMix for qRT-PCR, ThermoFisher Scientific). Real-time PCR Analysis was conducted with the 7900HT FAST real-time PCR detection system (Applied Biosystems). Taqman probes used are listed in Table 1. For high-throughput, real-time PCR analysis, samples were analyzed with the 96.96 Dynamic Array IFC on the Biomark HD system (Fluidigm).

TABLE 1
Taqman Probes

| Gene Name | Taqman Probe Thermo Fisher Catalog Number |
|---|---|
| Gapdh | Hs99999905_m1 |
| Kcna5 | Hs00969279_s1 |
| Gata4 | Hs00171403_m1 |
| Myocd | Hs00538071_m1 |
| Myog | Hs01072232_m1 |
| Myh7 | Hs01110632_m1 |
| Tead4 | Hs01125032_m1 |
| Mef2c | Hs00231149_m1 |
| Tbx18 | Hs01385457_m1 |
| Myh6 | Hs01101425_m1 |
| Smarcd3 | Hs00162003_m1 |
| Postn | Hs01566734_m1 |
| Pln | Hs00160179_m1 |
| Tnni3 | Hs00165957_m1 |
| Tnnc1 | Hs00268524_m1 |
| Srf | Hs00182371_m1 |
| Nppa | Hs00383231_m1 |
| Scn3a | Hs00165693_m1 |
| Esrrg | Hs00155006_m1 |
| Mesp1 | Hs00251489_m1 |
| Kcne1 | Hs00897540_s1 |
| Zfpm2 | Hs00201397_m1 |
| Col1a1 | Hs00164004_m1 |
| Ecm1 | Hs00189435_m1 |
| Ddr2 | Hs01025953_m1 |
| Cacna1c | Hs00167681_m1 |
| Atp2a2 | Hs00544877_m1 |
| Myh11 | Hs00224610_m1 |
| Actn2 | Hs00153809_m1 |
| Tnnt2 | Hs00943911_m1 |
| Col3a1 | Hs00943809_m1 |
| Kcnd2 | Hs01054873_m1 |
| Col5a2 | Hs00893878_m1 |
| Ryr2 | Hs00892883_m1 |
| Tbx5 | Hs00361155_m1 |
| Rpl19 | Hs02338565_gH |

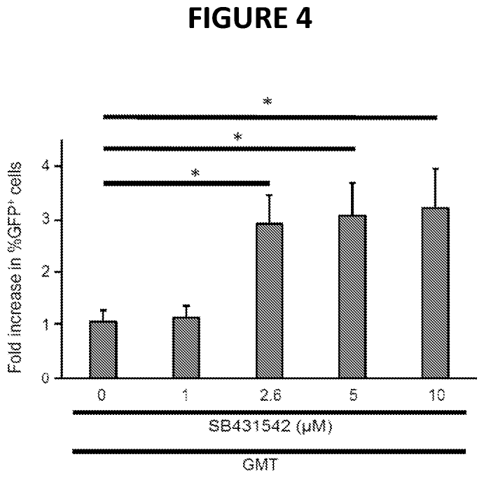

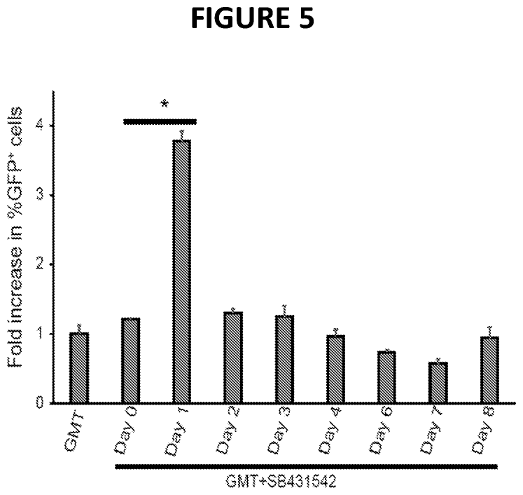

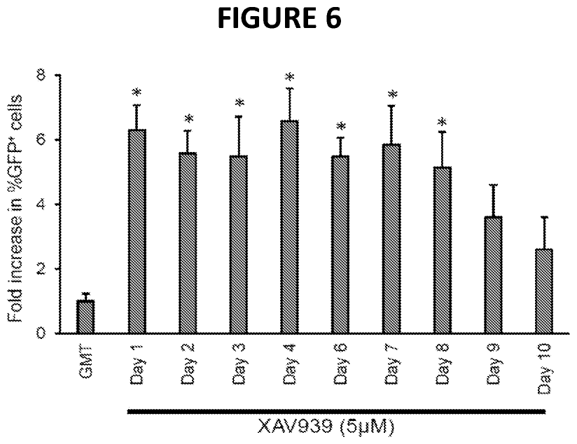

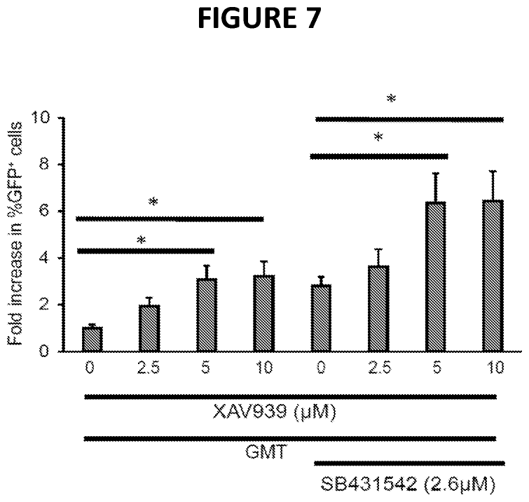

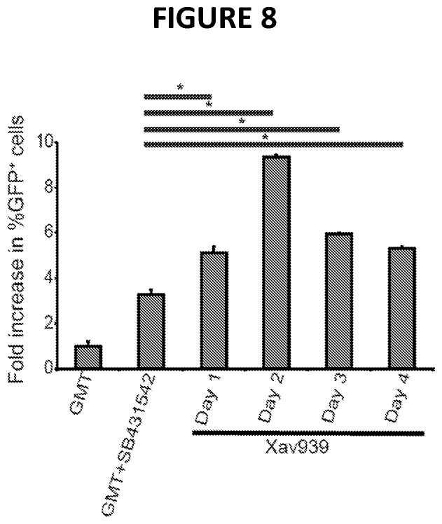

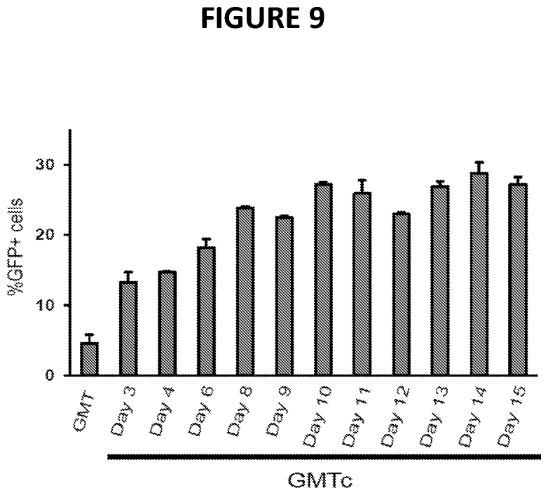

SB431542 was the most efficient of the TGF-β inhibitors at downregulating fibroblast gene expression and XAV939 was the most efficient of the WNT inhibitors at activating cardiac gene expression at 2 weeks of reprogramming. It was also found that SB431542 (2.6 μM) (FIG. 4) was most effective if added at day 1 of reprogramming (FIG. 5). Various doses and timing of XAV939 were tested to identify its optimal timing and concentration. It was found that 5 μM was the most effective dose and it had the same enhancement effect when added at any time during the first 8 days of reprogramming (FIG. 6). Upon combining the two small molecules, it was found that maximum reprogramming efficiency was achieved by adding XAV939 at day 2 of reprogramming, after adding SB431542 at day 1. (FIG. 7) This protocol resulted in a ˜nine-fold increase in cardiac reprogramming, as indicated by the increase in α-MHC-GFP+ iCMs (FIG. 8). Furthermore, after day 10 of reprogramming, the compounds were dispensable (FIG. 9).

### Example 2. SB431542 and XAV939 Increases the Efficiency and Quality of iCMs In Vitro

After validating and optimizing the timing, dose, and conditions for adding small molecules to the reprogramming cocktail, GMT-overexpressing fibroblasts were treated with SB431542 (2.6 μM) at day 1 and XAV939 (5 μM) at day 2. For FACS analysis of GFP expression, reprogrammed cardiac fibroblasts were harvested from cultured dishes and analyzed on the LSR-II (BD) with FlowJo software. For live-cell sorting and analysis, cells were dissociated using TRYPLE (Thermo Fisher) and sorted using an LSRII (BD) machine. For fixed-cell FACS, cells were fixed with formalin for 15 min, permeabilized with 0.1% v/v Triton X-100, and stained with anti-GFP (Thermo Fisher) and either anti-α-Sarcomeric Actinin (Sigma) or anti-cardiac myosin 2 (Thermo Fisher) antibodies, followed by secondary antibodies conjugated with Alexa-594 (Thermo Fisher).

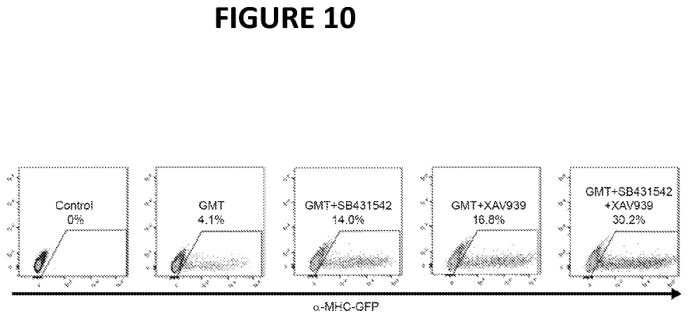

The combination of SB431542 (2.6 μM) at day 1 and XAV939 (5 μM) at day 2 increased reprogramming efficiency to ˜30% in cardiac fibroblasts within two weeks (FIG. 10). With SB431542 alone, cells matured faster with beating cells appearing as early as 3 weeks after GMT infection. Remarkably, the combination of SB431542 and XAV939 resulted in appearance of beating cells as early as 1 week, compared to 6-8 weeks with GMT alone. Troponin T staining and advanced sarcomere organization was also observed in iCMs after only 2 weeks of reprogramming with GMT+SB431542/XAV939 versus 4-6 weeks with GMT alone.

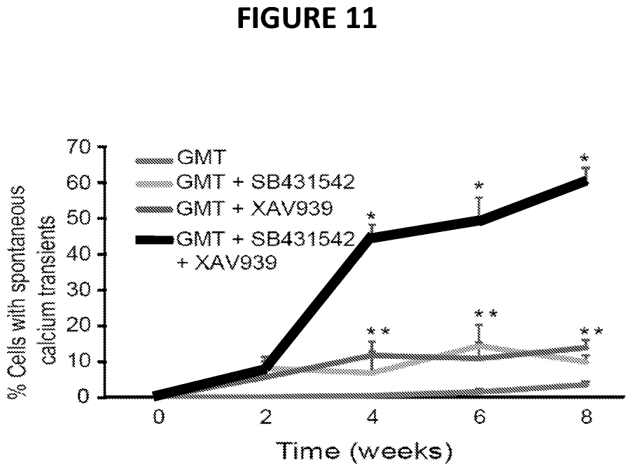

Calcium transients were assessed as previously described. Qian, L., et al. (2012) Nature 485:593-598. Briefly, isolated myocytes were loaded with Fluo-4 for 30 min at room temperature before being transferred to the superfusion chamber. The loading solution contained a 1:10 mixture of 5 mM Fluo-4 AM in dry DMSO and Powerload™ concentrate (Invitrogen), which was diluted 100-fold into extracellular Tyrode's solution containing suspended myocytes. An additional 20 min was allowed for de-esterification before recordings were taken. Contractions and calcium transients were evoked by applying voltage pulses at 1 Hz between platinum wires placed on either side of the cell of interest and connected to a field stimulator (IonOptix, Myopacer). Fluo-4 fluorescence transients were recorded via a standard filter set (#49011 ET, Chroma Technology). Resting fluorescence was recorded after cessation of pacing, and background light was obtained after picking up and removing the cell from the field of view at the end of the experiment. Contractions were optically recorded simultaneously with calcium transients by illuminating the cell of interest in bright-field subsequently directed to a CCD camera (IonOptix Myocam). The cell-length signals were converted to voltage via a video motion director (VED 205; Crescent Electronics) and contraction amplitudes from different myocytes were normalized by calculating the percent change in cell length. These studies revealed that over ˜50% of the cells possessed spontaneous calcium transients within 4 weeks of reprogramming in the presence of the two compounds (FIG. 11).

Gene expression profiles in iCMs 3, 5 and 6 weeks after cardiac reprogramming were also examined using RNAseq. For these experiments, α-MHC-GFP+ in vitro mouse iCMs, TNT-GFP+ human iCMs, and control dsRed infected cardiac fibroblasts (mock) were sorted with a FACS Aria II cell sorter. For live-cell sorting for RNAseq, α-MHC-GFP mouse cardiac fibroblasts or hTNT-GFP+ reprogrammed human cardiac fibroblasts were dissociated from cultured dishes and sorted for GFP+ expression with an Aria II FACS sorter. After Langendorff isolation of the in vivo reprogrammed iCMs, the iCMs were picked based on their expression of periostin cre YFP reporter using a micropipette. Their RNA was isolated using the Qiagen, miRNeasy Micro Kit #210874. Using the Ovation RNA-seq System v2 Kit (NuGEN), the total RNA (20-50 ng) was reverse transcribed to synthesize first-strand cDNA using a combination of random hexamers and a poly-T chimeric primer. The RNA template was then partially degraded by heating and the second-strand cDNA was synthesized using DNA polymerase. double-stranded DNA was then amplified using single primer isothermal amplification (SPIA). SPIA is a linear cDNA amplification process in which RNase H degrades RNA in DNA/RNA heteroduplex at the 5′-end of the double-stranded DNA, after which the SPIA primer binds to the cDNA, and the polymerase starts replication at the 3′-end of the primer by displacing the existing forward strand. Random hexamers were then used to linearly amplify the second-strand cDNA. cDNA samples were fragmented to an average size of 200 bp using the Covaris S2 sonicator. Libraries were made from the fragmented cDNA using the Ovation Ultralow V2 kit (NuGen). Following end repair and ligation, the libraries were PCR amplified with 9 cycles. Library quality was assessed by a Bioanalyzer on High-Sensitivity DNA chips (Agilent) and concentration was quantified by qPCR (KAPA). The libraries were sequenced on the HiSeq 2500 sequencer with a single-read, 50-cycle sequencing run (Illumina). The RNAseq-analysis pipeline was utilized as reported previously. Theodoris, C. V., et al. (2015) Cell 160:1072-1086.

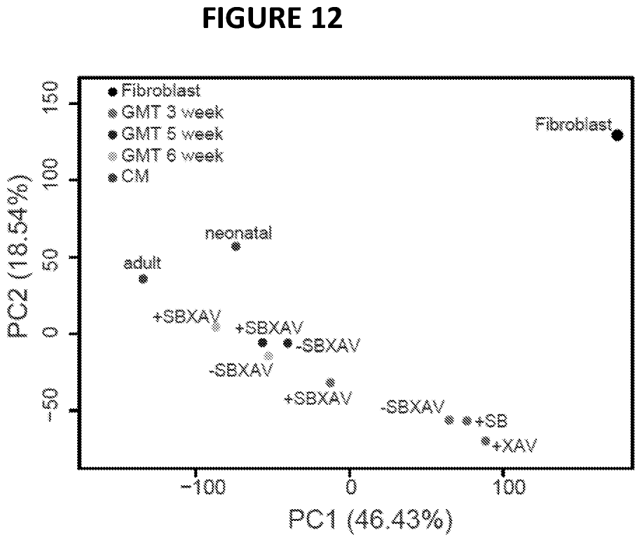

Only iCMs treated with SB431542 and XAV939 (GMTc), but not iCMs treated with single compound, for 3 weeks had a more cardiomyocyte-like transcriptional profile as reflected by principal component analysis (PCA) and heat maps for the top differentially expressed genes (FIG. 12). Reprogramming with GMTc for 5 or 6 weeks resulted in gene-expression profiles closer to adult mouse ventricular cardiomyocytes than reprogramming with GMT alone (FIG. 12). Addition of SB431542 and XAV939 (+SBXAV) to GMT-transduced fibroblasts resulted in an advanced state of reprogramming, closer to the cardiomyocyte (CM) state, compared to GMT alone (-SBXAV). Neither SB431542 (+SB) or XAV939 (+XAV) alone with GMT showed this effect.

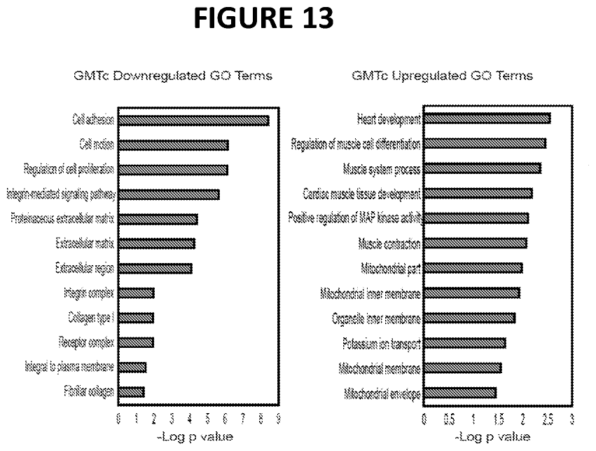

Furthermore, iCMs reprogrammed with GMTc for 3, 5 or 6 weeks significantly downregulated genes with gene ontology (GO) terms related to TGF-β and WNT signaling, similar to cardiomyocytes. Comparing gene expression profiles between GMT and GMTc at 5 weeks, it was found that the top differentially regulated GO terms were related to regulation of extracellular matrix, ion channels, and muscle formation (FIG. 13). Upon closer analysis of the most highly expressed cardiac-contractile, ion-handling, and extracellular-matrix genes, it was found that the compounds brought the expression of these genes very close to that of adult cardiomyocytes, albeit not completely. Thus, inhibiting TGF-β and WNT signaling increased both the quality and speed of cell-fate conversion in vitro.

### Example 3. SB431542 and XAV939 Enhance Cardiac Reprogramming by Inhibiting Canonical TGF-β and WNT Signaling

Next, the mechanisms by which the small molecules regulate TGF-β and WNT signaling were investigated. Proteins from cell lysates containing 20 μg of protein were separated by SDS-PAGE and electrophoretically transferred onto polyvinylidene difluoride membranes (BioRad). Membranes were then washed in PBS, treated with blocking buffer (Li-cor), and then incubated with primary antibodies at 4° C. overnight. The following primary antibodies were used: anti-GAPDH antibody loading control (Abcam # ab9484, 0.5 μg/ml), anti-Active β-Catenin antibody (Millipore #05-665, 1:1000), anti-T-Antigen (Abcam # ab16879, 1:1000), and anti-phospho-Smad2/3 (Cell Signaling #8685S, 1:1000). Membranes were washed in PBS and incubated with the appropriate secondary antibodies (Li-Cor, IRDye 600LT; IRDye 800CW, 1:10,000) for 1 hour at room temperature. Membranes were washed and visualized with an Odyssey Fc Dual-mode Imaging System.

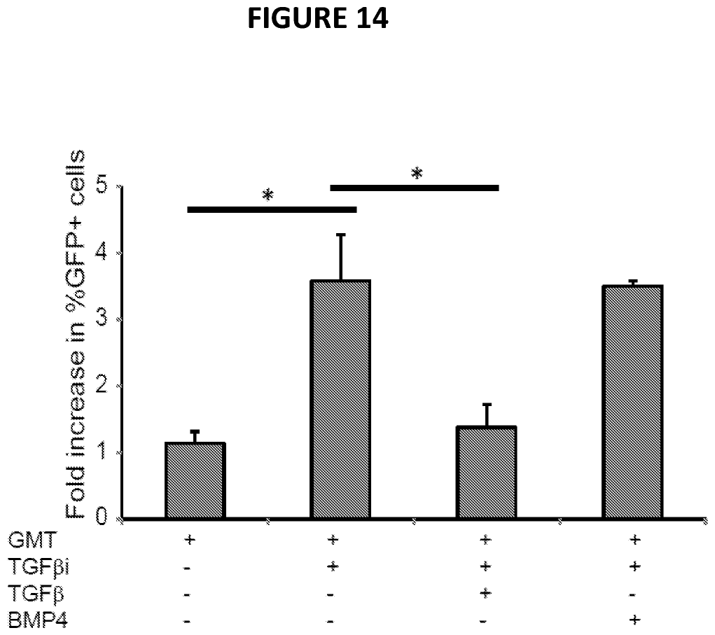

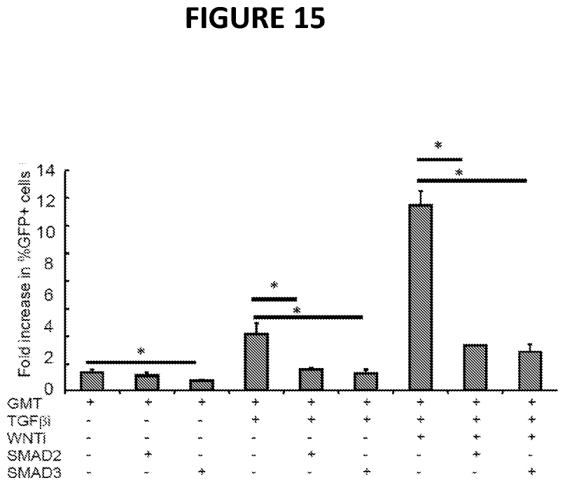

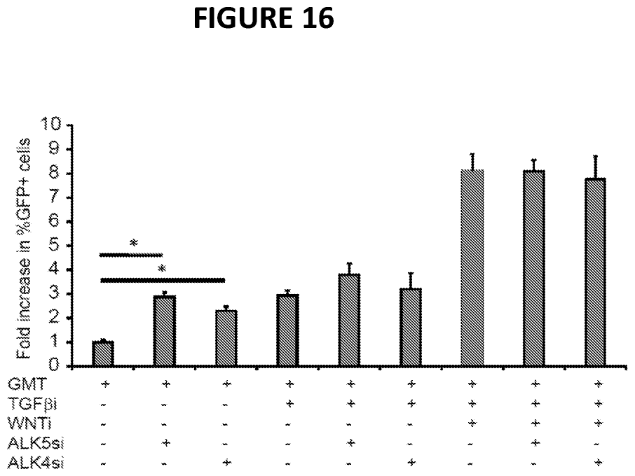

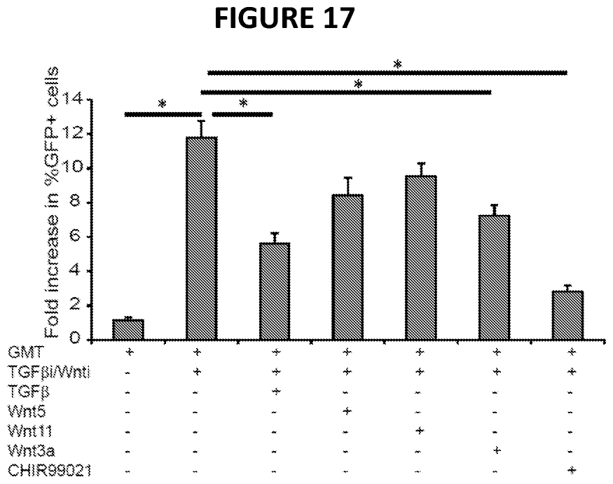

The phosphorylation of SMAD2/3 (indicator of TGF-β activation) and expression of active β catenin (indicator of WNT activation) were significantly reduced by SB431542 or XAV939. It was also observed that activating TGF-β or canonical WNT signaling reversed the reprogramming efficiency gains induced by SB431542 or XAV939, respectively. Adding excess TGF-β1 ligand during reprogramming reversed the effect of SB431542 and partially inhibited the combined effect of SB431542 and XAV939; however, BMP4, which regulates other aspects of TGF-β signaling, did not significantly affect cardiac reprogramming (FIG. 14). Moreover, overexpression of constitutively active SMAD2 or SMAD3 (TGF-β signaling effectors) abolished cardiac reprogramming enhancement by SB431542 (FIG. 15). Because SB431542 specifically inhibits activin receptor-like kinase (ALK) 4 and ALK5 receptors, the effect of knocking down ALK4 or ALK5 with siRNA on direct cardiac reprogramming was tested. An ˜80% reduction of ALK4 or ALK5 (FIG. 16) enhanced direct cardiac reprogramming, similar to SB431542 (FIG. 16). Similarly, WNT3a, which activates canonical WNT signaling, partially blocked the effects of SB431542 and XAV939 on cardiac reprogramming; however, activating the non-canonical WNT pathway through WNT5 or WNT11 did not have a significant effect. Furthermore, adding the glycogen synthase kinase 3β (GSK3β) inhibitor CHIR99021, which activates the canonical WNT pathway, reversed the combined effect of SB431542 and XAV939 (FIG. 17). Thus, SB431542 and XAV939 enhance cardiac reprogramming by inhibiting TGF-β and canonical WNT signaling.

### Example 4. TGF-β and WNT Inhibitors Enhance Cardiac Reprogramming In Vivo

It was previously shown that intramyocardial injection of GMT successfully reprogrammed fibroblasts into iCMs in vivo, increased heart function and decreased scar size after injury, but did not completely restore cardiac function. Qian, L., et al. (2012) Nature 485:593-598. The effects of inhibiting TGF-β and WNT signaling on cardiac reprogramming in vivo were tested by injecting SB431542 (10 mg/kg/day) and XAV939 (2.5 mg/kg/day) intraperitoneally every day for 2 weeks after coronary ligation and intramyocardial injection of GMT-encoding retrovirus (GMTc).

For the animal studies, Postn-Cre:R26R-YFP mice were obtained by crossing Postn-Cre mice and Rosa26-EYFP mice. The animal protocol for surgery was approved by the University of California, San Francisco Institutional Animal Care and Use Committee. All surgeries were performed as previously described. Qian, L., et al. (2012) Nature 485:593-598. Briefly, mice were anaesthetized with 2.4% isoflurane/97.6% oxygen and placed in a supine position on a heating pad (37° C.). Animals were intubated with a 19 G stump needle and ventilated with room air using a MiniVent Type 845 mouse ventilator (Hugo Sachs Elektronik-Harvard Apparatus; stroke volume, 250 μl; respiratory rate, 120 breaths per minute). Myocardial infarction (MI) was induced by permanent ligation of the left anterior descending (LAD) artery with a 7-0 prolene suture as described in Qian, L., et al. (2012) Nature 485:593-598. Sham-operated animals served as surgical controls and were subjected to the same procedures as the experimental animals with the exception that the LAD was not ligated. A pool of concentrated virus (GMT) was mixed and 10 μl of mixed virus and 10 μl of PBS were injected into the myocardium through an insulin syringe with an incorporated 29-G needle (BD). Injection with a full dosage was carried out along the boundary between the infarct zone and border zone based on the blanched infarct area after coronary artery occlusion. At days 2 through 15, the animals received daily intraperitoneal injections of SB431542 (10 mg/kg/day) and XAV939 (2.5 mg/kg/day).

Serial echocardiography was conducted before MI and 1, 2, 4, 8 and 12 weeks after MI to assess the cardiac function. Echocardiography was performed with the Vevo 770 High-Resolution Micro-Imaging System (VisualSonics) with a 15-MHz linear-array ultrasound transducer. The left ventricle was assessed in both parasternal long-axis and short-axis views at a frame rate of 120 Hz. End-systole or end-diastole were defined as the phases in which the left ventricle appeared the smallest and largest, respectively, and used for ejection-fraction measurements. To calculate the shortening fraction, left-ventricular end-systolic and end-diastolic diameters were measured from the left ventricular M-mode tracing with a sweep speed of 50 mm/s at the papillary muscle. B-mode was used for two-dimensional measurements of end-systolic and end-diastolic dimensions.

At the end of the experiments (12 weeks after MI), animals were exposed to magnetic resonance imaging (MRI) to assess cardiac structure and function. In vivo MRI imaging was performed with a 7T preclinical horizontal bore magnet interfaced with an Agilent imaging console. Animals were anaesthetized by inhalation of 2% isoflurane with 98% oxygen and placed into a home-built, linear-polarized birdcage coil with 28-mm internal diameter (ID). Body temperatures during imaging experiments were kept at 34° C. Heartbeats, breading, and temperature of the animals were monitored with an MRI-compatible, small-animal life-support system (SA Instruments Inc, Stony Brook, N.Y.). Location and long and short axes of mouse hearts were determined from scout images with the following parameters: Repetition time (TR), 10 msec; echo time (TE), 4 msec; excitation flip angle, 20-degree; field of view (FOV), 6 cm²; matrix dimension, 128×128; slice thickness, 1 mm; number of repetitions, 2. To evaluate functional parameters of control and infarcted hearts, MRI images of short axes were acquired with a spin echo-pulse sequence. Parameters of the acquisition were TR=1 sec, TE=10 msec, in-plane image resolution=200 □m, and acquisition time=˜20 min (depended on heart rate). Cardiac and breathing gating were employed. To measure the ejection fraction, nine or ten short-axis slices of the heart were acquired at diastole (zero delay after R-heart-peak) and systole (45% of the R-R interval delay from the R-peak).

This assessment was followed by sacrificing the animals and harvesting the hearts for histological studies. Standard Masson's Trichrome staining was performed on hearts 12 weeks post-viral delivery and coronary artery ligation. To determine scar size, ImagePro software was used to measure the scar area (blue) and healthy area (red) on transverse sections spanning four levels within the left ventricle of an MI heart. From each level, four slices of tissues were measured as technical quadruplicates (for a total of 16 sections).

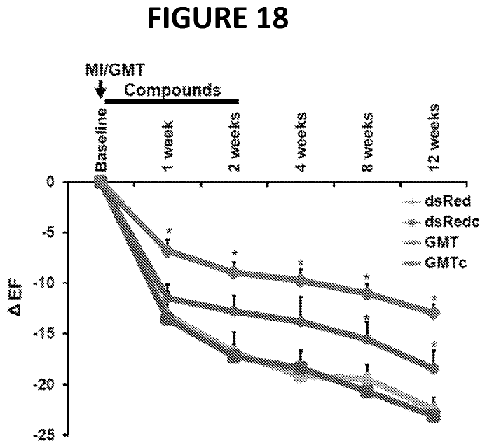

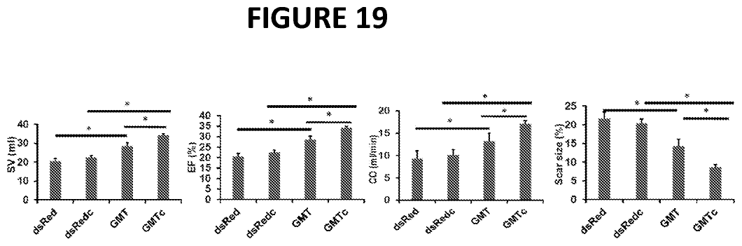

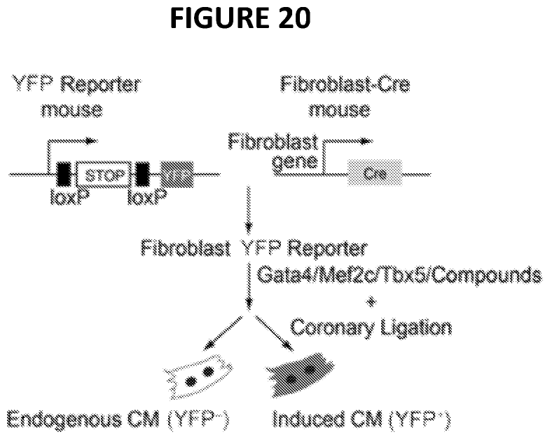

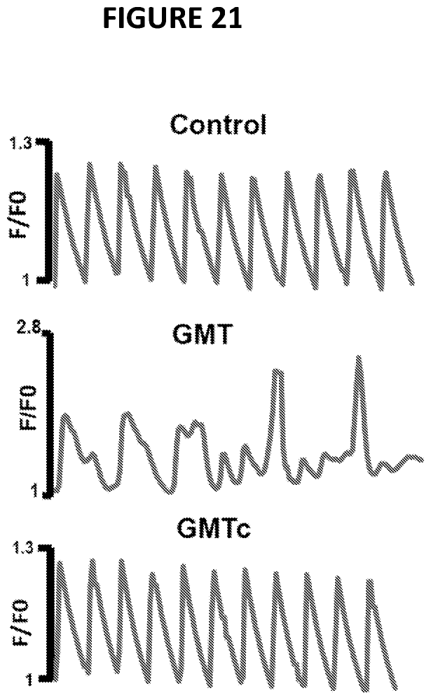

GMTc significantly enhanced cardiac function compared to treatment with GMT alone, as reflected by changes in the ejection fraction (EF) assessed by echocardiography (FIG. 18). Treatment with GMTc significantly preserved cardiac function compared to animals treated with GMT alone. The improved function occurred as early as 1 weeks after MI, consistent with the in vitro observations showing an increased reprogramming speed. The inhibitors alone did not significantly affect cardiac function. At 12 weeks after MI, blinded magnetic resonance imaging (MRI) were conducted to evaluate heart structure and function, as it is the most accurate form of measurement. Thick muscle within the infarct region was observed only in the group treated with GMTc, even at the apex of the heart. Heart function revealed by MRI was significantly improved in animals treated with GMTc compared to GMT alone, as assessed by changes in stroke volume (SV), EF, and cardiac output (CO) (FIG. 19).

Histological analyses were performed to quantify the scar size and detect the presence of muscle within the infarct area of treated hearts. Consistent with the in vivo imaging observations, it was found that threads of myocytes developed within the infarct site of hearts treated with GMT alone, similar to previous report; however, in hearts isolated from animals treated with GMTc, thick bands of myocytes were observed within the infarct zone. To confirm that this remuscularization was due to reprogramming, ROSA-Lox-Stop-Lox-YFP mice were crossed with PeriostinCre mice to generate ROSA-YFP/Periostin-Cre lineage-tracing mice. These mice express YFP only in fibroblasts and their descendants and, therefore, distinguish fibroblast-derived cardiomyocytes (FIG. 20) from endogenous cardiomyocytes. It was found that the remuscularization around the infarct area was due to newly formed iCMs, as these cells stained positive for troponin T and the YFP reporter. However, no YFP cells were found in the control groups or in areas distal to the infarct site.

To assess the functionality and quality of the in vivo iCMs, a Langendorff preparation was used to isolate iCMs from ROSA-YFP/Periostin-Cre mice. Adult cardiomyocytes (CMs) were isolated as described in Qian, L., et al. (2012) Nature 485:593-598, with minor modifications. Briefly, adult mice were anaesthetized with isoflurane and mechanically ventilated. Hearts were removed and perfused retrogradely via aortic cannulation with a constant flow of 3 ml/min in a Langendorff apparatus. Hearts were perfused at 37° C. for 5 min with Wittenberg Isolation Medium (WIM) containing 116 mM NaCl, 5.4 mM KCl, 6.7 mM MgCl2, 12 mM glucose, 2 mM glutamine, 3.5 mM NaHCO3, 1.5 mM KH2PO4, 1.0 NaH2PO4, 21 HEPES, with 1.5 nM insulin, essential vitamins (GIBCO), and essential mM amino acids (GIBCO) (pH 7.4), followed by digestion solution (WIM supplemented with 0.8 mg/ml collagenase II and 10 μM CaCl2) for 10 min (4 min for paired CMs that were used in cell-cell coupling experiments). Hearts were then removed from the Langendorff apparatus while intact (with tissues loosely connected). Desired areas (i.e., border/infarct zone) were then micro-dissected under the microscope, mechanically dissociated, triturated, and resuspended in a low-calcium solution (WIM supplemented with 5 mg/ml bovine serum albumin, 10 mM taurine, and 150 μM CaCl2). Cells were then spun at low speed, supernatant was removed, and calcium was gradually reintroduced through a series of washes. For electrophysiology experiments, cells were used on the same day as isolation and, until recordings, were stored at room temperature (21° C.) in M199 (Gibco) supplemented with 5 mM creatine, 2 mM 1-carnitine, 5 mM taurine, and 1.5 nM insulin. For immunohistochemistry, cells were plated onto laminin-coated culture slides, allowed to adhere, and fixed on the day of isolation. For RNAseq, contractility analysis, and calcium-transients measurements, iCMs were selected manually by micro-pipette based on the presence of periostin-Cre:R26R-YFP signal under the fluorescent microscope immediately after isolation.

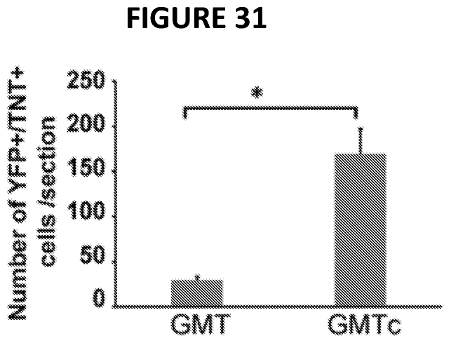

The number of YFP+ iCMs significantly increased by fivefold in animals treated with GMTc compared to GMT alone. (FIG. 31) All iCMs isolated from either GMT- or GMTc-treated mice were rod shaped and formed well-organized sarcomere structures, similar to adult cardiomyocytes. In terms of contractility, all cells isolated from GMT-treated mice exhibited spontaneous calcium transients, but over 70% of the cells isolated from GMTc mice did not beat spontaneously. However, they synchronized with external electrical stimulation, indicating a more mature phenotype (FIG. 31).

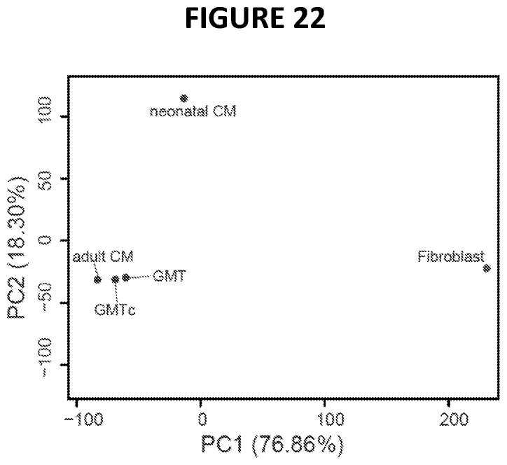

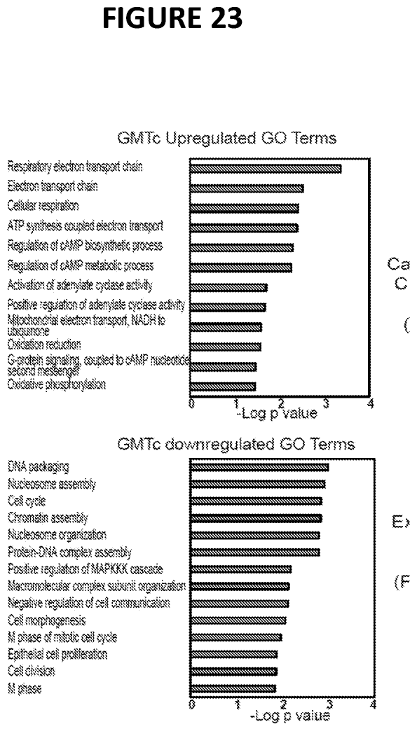

RNA-seq was also conducted to compare whole-transcriptome changes between control cardiomyocytes, GMT iCMs, and GMTc iCMs isolated from in vivo reprogrammed hearts. The gene expression of GMTc iCMs was more similar to adult ventricular cardiomyocytes than GMT iCMs, but different from neonatal cardiomyocytes, as reflected by PCA analysis (FIG. 22). Additionally, GMTc iCMs displayed more fully downregulated genes with GO terms related to TGF-β and WNT signaling, similar to adult cardiomyocytes. GO analysis for the genes that were differentially regulated by at least two-fold between GMT and GMTc iCMs revealed downregulation of genes involved in cell division and mitosis in GMTc, as well as upregulation of metabolic genes and cAMP related genes consistent with a more mature phenotype (FIG. 23). Focusing on changes in highly expressed major cardiac and fibroblast genes, it was found that GMTc iCMs displayed a more complete upregulation of cardiac genes and downregulation of fibroblast genes compared to GMT iCMs.

### Example 5. TGF-β and WNT Inhibitors Enhance Reprogramming of Human Adult Cardiac Fibroblasts

It was previously reported that GMT alone is insufficient to reprogram human fibroblasts, but found that ESSRG, MYOCD, ZFPM2, and MESP1 in combination with GMT (7F) can reprogram human cells to similar quality as in mice. Fu, J. D., et al. (2013) Stem Cell Reports 1:235-247. Here, the effects of adding SB431542 and XAV939 to human cardiac reprogramming induced by 7F were tested.

Ventricular normal human cardiac fibroblasts (HCF) were purchased from Lonza. In order to use the most relevant type of fibroblasts, an reversibly immortalized cell line of adult human cardiac fibroblasts was generated with a floxed T-antigen. To generate the stable immortalized cell line, the cells were infected with floxed human T-antigen lentivirus, which contains a puromycin-selection cassette (Addgene plasmid #18922). Cells were selected using 1 μg/ml puromycin for 5 days. Cells were then infected with Cre virus at the day of reprogramming to cut out the T-antigen. Reprogramming was conducted as previously described. Fu, J. D., et al. (2013) Stem Cell Reports 1:235-247. Briefly, pMXs retroviral vectors encoding the seven human cardiac developmental factors (Gata4, Mef2c, Tbx5, Myocardin, Esrrg, Mesp1, and Znfpm2) were transfected into Platinum-A (Cell Biolabs) cells to generate viruses. After 48 h, HCFs were transduced overnight with the pool of virus containing supernatants and supplemented with 6 μg/ml polybrene. Media was then replaced with iCM media [DMEM:M199 (4:1), 10% FBS, 1× non-essential amino acids (NEAA), 1× penicillin/streptomycin] containing SB431542 (2.6 μM) at 24 h post-infection and XAV939 (5 μM) 48 h post-infection. For visualization of reprogramming and cell sorting, reprogrammed HCFs were either transduced with plx-hTNT-GFP or plx-hTNT-GCaMP5. Briefly, plx-hTNT-GFP or plx-hTNT-GCaMP5 were transfected into HEK 293FT cells with Fugene HD along with the lentivirus packaging plasmids pMD2.G and psPAX2 to generate the lentivirus. After 48 h, HCFs were transduced overnight and supplemented with 6 μg/ml polybrene. At day 4 of reprogramming, RPMI1640 with B27 supplement was added every 3 days at 25% increments with iCMs until it replaced iCM media completely at day 15.

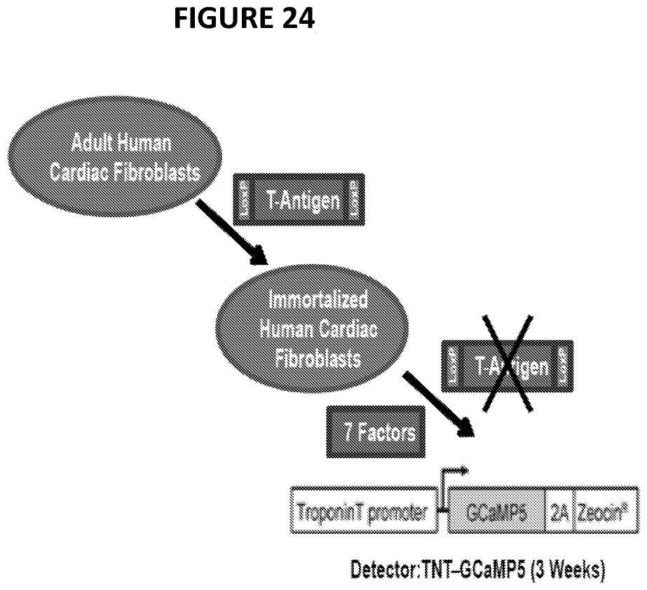

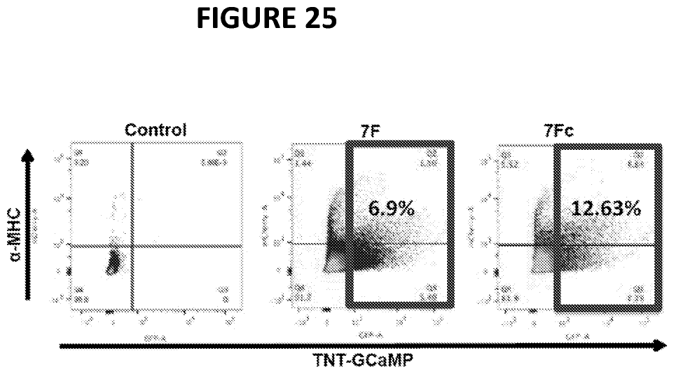

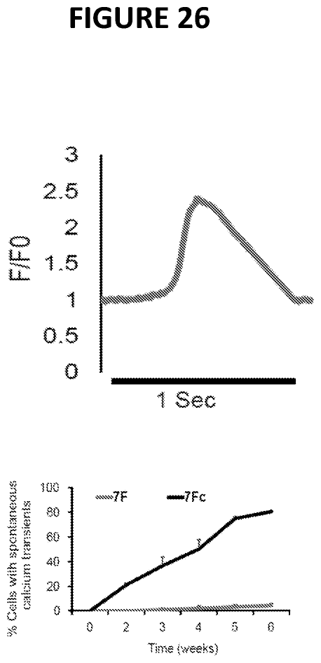

Using the TNT-GCaMP reporter to detect cardiac reprogramming (FIG. 24), it was found that 7F plus SB431542 and XAV939 (7Fc) doubled the percentage of iCMs reprogrammed by 7F (FIG. 25). iCMs reprogrammed with 7Fc also exhibited sarcomere formation as early as 3 weeks. Furthermore, calcium sparks occurred after just 10 days of reprogramming with 7Fc, and within 3 weeks of reprogramming, these calcium transients became more homogenous throughout the cell. Within 4 weeks, over 50% of 7Fc reprogrammed exhibited spontaneous calcium transients compared to less than 5% with 7F, potentially indicated their maturity (FIG. 26).

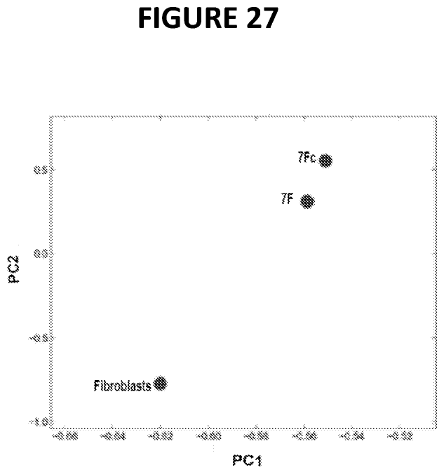

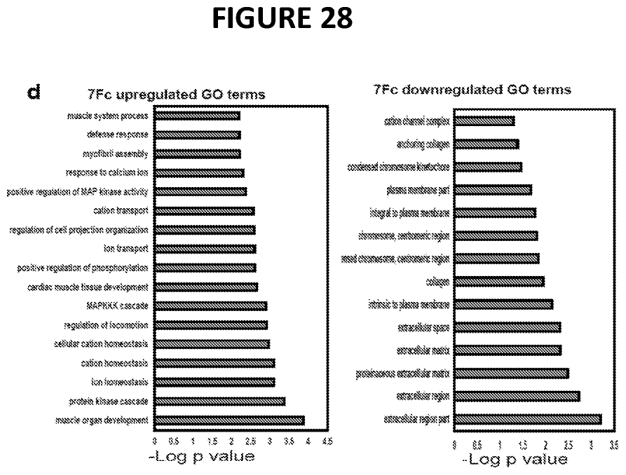

To assess the quality of reprogrammed cells, RNA-seq was performed after 4 weeks of 7F-induced reprogramming with or without chemicals. Indeed, the gene-expression profile changes in 7Fc iCMs was accelerated compared to 7F iCMs, as indicated by PCA (FIG. 27). The reprograming with the 4FC produced similar results to reprogramming using 7Fc and TNT-GFP as a reporter. Furthermore, gene expression of the WNT and TGF-β signalling pathways was significantly downregulated in 7Fc iCMs compared to 7F iCM.s. GO analysis was performed of the genes that were differentially regulated between the 7F and 7Fc iCMs by at least two-fold and it was found that these genes were mostly involved in downregulation of extracellular-matrix formation, and collagens, as well as upregulation in calcium, and ion transports related genes, similar to what was found in mouse reprogramming. (FIG. 28) By looking more closely at major cardiac and fibroblast genes, it was found that the chemicals significantly enhanced expression of cardiac genes and suppressed the expression of fibroblast genes. The reprograming with the 4FC produced similar results to reprogramming using 7Fc and TNT-GFP as a reporter.

Together these data show that inhibiting TGF-β and WNT signaling with small molecules significantly enhanced cardiac reprogramming of postnatal mouse and human cardiac fibroblasts in vitro and in vivo.

Surprisingly, it was also discovered that TGF-β signaling is essential for direct cardiac reprogramming by inhibiting this pathway with SB431542, a small molecule that selectively inhibits ALK5 (the TGF-β type I receptor), ALK4, and ALK7.

Interestingly, the RNA-seq data revealed that in vitro and in vivo GMTc iCMs have a gene expression profile more consistent with adult ventricular cardiomyocytes rather than neonatal cardiomyocytes. Surprisingly, the RNA expression levels of certain cardiac genes in GMTc iCMs in vitro and in vivo were actually higher than in isolated control endogenous cardiomyocytes (e.g. TNNT, ACTC1, ACTN2, MYH7 and RYR2). This unexpected observation could reflect that iCMs are still undergoing reprogramming and therefore are actively transcribing higher levels of these lineage-specific genes to support their cell-fate transformation. Alternatively, these genes may represent targets that are particularly sensitive to the sustained activity of exogenous GMT. Nevertheless, GMTc efficiently downregulated most of the fibroblast genes in vitro and in vivo to similar levels as adult ventricular cardiomyocytes.

### Example 6. Reprogramming of Human Fibroblasts with Fewer Factors than the Conventional 7F System

In order to determine whether any one or combination of the 7F reprogramming factors previously reported as necessary for sufficient transdifferentiation of human fibroblasts into cardiomyocytes were non-essential, reprogramming was assessed with single factors and various combinations of multiple factors removed.

First, removal of a single factor in the 7Fc was tested to see if it could be removed and still fully reprogram human cardiac fibroblasts. By removing one factor at a time it was found that reprogramming of human fibroblasts without Mesp1, ZFPM2, or ESSRG resulted in similar gene expression profiles as compared to the 7Fc reprogramming method. In addition, when different combination of two factors were removed, it was found that similar gene expression profiles resulted from reprogramming in the presence of SB431542 and XAV939 without the combinations of: (1) Zfpm2 and Esrrg and (2) Zfpm2 and Mesp1. On the other hand, Myocardin appeared to be of particular importance for efficient reprogramming of human fibroblasts to cardiomyocytes. Removal of Myocardin from the reprogramming cocktail resulted in lower expression of cardiomyocyte genes and increased residual expression of fibroblasts genes.

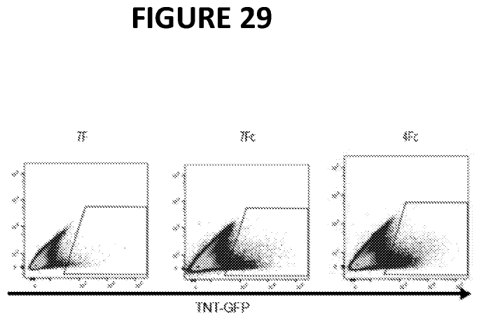

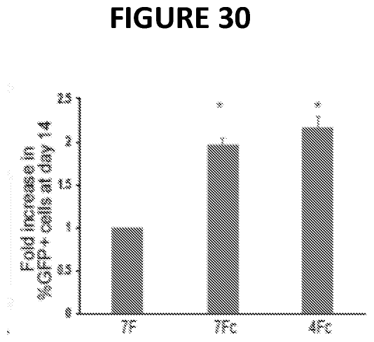

Surprisingly, it was also found that the same degree of reprogramming could be achieved with just 4 factors: GATA4, MEF2C, TBX5, and Myocardin when reprogrammed in the presence of SB431542 and XAV939 (4Fc). FACS analysis showed that a similar percentage of TNT+ iCMs were obtained using the 4Fc method and immunofluorescence revealed highly organized sarcomere organization in 4Fc and 7Fc induced iCMs (FIG. 29, 30). In particular, removal of Mesp1, Zfpm2, and Esrrg from the reprogramming factor cocktail results in similar expression levels of cardiomyocyte genes as compared to human fibroblasts reprogrammed with 7Fc reprogramming method.

Remarkably, in the presence of SB431542 and XAV939 it was discovered that human fibroblasts could efficiently be reprogrammed to cardiomyocytes with just a three factor combination, Myocardin, Mef2c, and Tbx5, (MMT). The combination of MMT is sufficient to induce cardiac reprogramming in the presence of SB431542 and XAV939, as indicated by increased expression of the TNT-GFP reporter. The addition of an anti-inflammatory reagent (dexamethasone) was also found to enable MMT reprogramming in the presence of SB431542 and XAV939 (data not shown). In addition, the combination of MMT and compounds resulted in the appearance of Troponin-T positive cells, having advanced sarcomere organization within three weeks of reprogramming.

### Example 7: Use of siRNA with Dexamethasone in MMTc Reprogramming

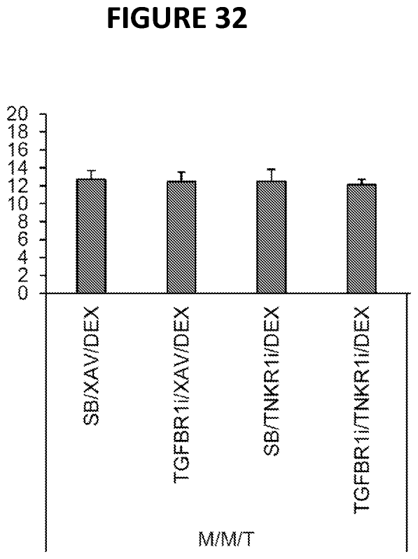

The ability for siRNA molecules to replace the small molecule chemical inhibitors of MMTc was tested using an siRNA to TGF-β Receptor 1 and Tankyrase 1. The use of the small interfering RNAs was shown to be comparably efficient in the reprogramming of the human fibroblasts into iCMs, with the siRNA to siRNA to TGF-β Receptor 1 and Tankyrase 1 being essentially equivalent in reprogramming activity to SB431542 and XAV939, respectively (FIG. 32).

Briefly, 20000 cells/well were transfected using RNAiMAX at day at a concentration of 100 nM siRNA/well at Day −1 of reprogramming, and the transfection reagent was removed 24 hours later. Retroviruses encoding MMT infected Human Cardiac Fibroblasts at day 0 of the experiments and removed 24 hours later. Reprogramming was then conducted as previously described. Mohamed TMA et al., Circulation. 2017; 135:978-995. Briefly, a cocktail of three separate retroviruses vectors, each encoding one of the three reprogramming factors, was used to infect HCFs at day 1, and excess virus removed 24 hours later. The retrovirus remains integrated into the genome for the full period of the experiment. SB431542 (2.6 uM) was added at day 1 till day 8, XAV9393 (5 uM) was added at day 2 till day 8, and Dexamethasone (100 nM) was added day 1 till day 21.

The examples set forth above provide the first simple cocktail, using either small-molecules or siRNA, with the ability to enhance direct cardiac reprogramming using reprogramming factors in postnatal human and mouse cardiac fibroblasts in vitro and in vivo. These data show that a TGF-β inhibitor and a WNT inhibitor significantly improved cardiac function and reduced the number of reprogramming factors needed for human reprogramming from seven to four or to as few as three factors.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where the features or aspects of the invention are described in terms of Markush groups, those skilled in the art will recognize that the invention is also thereby described in terms of any individual member or subgroup members of the Markush group.

All publications, patent applications, patents and other references mentioned herein are expressly incorporated by reference in their entirety, to the same extent as if each were incorporated by reference individually. In case of conflict, the present specification, including definitions, will control.

What is claimed is:

1. A method for generating an induced cardiomyocyte or cardiomyocyte-like cell, the method comprising: introducing to a non-cardiomyocyte an effective amount of a WNT inhibitor and one or more reprogramming factors, thereby generating an induced cardiomyocyte or a cardiomyocyte-like cell.

2. The method of claim 1, wherein the WNT inhibitor is a small molecule.

3. The method of claim 1, wherein the WNT inhibitor is an siRNA.

4. A method for generating an induced cardiomyocyte or cardiomyocyte-like cell, the method comprising: introducing to a non-cardiomyocyte an effective amount of a TGF-β inhibitor and one or more reprogramming factors, thereby generating an induced cardiomyocyte or a cardiomyocyte-like cell.

5. The method of claim 4, wherein the TGF-β inhibitor is a small molecule.

6. The method of claim 4, wherein the TGF-β inhibitor is an siRNA.

7. A method for generating an induced cardiomyocyte or cardiomyocyte-like cell, the method comprising: introducing to a non-cardiomyocyte an effective amount of a WNT inhibitor, an effective amount of a TGF-β inhibitor, and one or more reprogramming factors, thereby generating an induced cardiomyocyte or a cardiomyocyte-like cell.

8. The method of claims 1-8, wherein the non-cardiomyocyte is a human cell.

9. The method of claim 7 or 8, wherein the WNT inhibitor is a small molecule.

10. The method of claim 7 or 8, wherein the WNT inhibitor is an siRNA.

11. The method of claims 7-10, wherein the TGF-β inhibitor is a small molecule.

12. The method of claims 7-10, wherein the TGF-β inhibitor is an siRNA.

13. The method of claims 7-12, wherein the TGF-β inhibitor is administered prior to administration of the WNT inhibitor.

14. The method of claims 7-12, wherein the TGF-β inhibitor is administered concurrently with administration of the WNT inhibitor.

15. The method of any one of claims 1-14, wherein the non-cardiomyocyte is selected from the group consisting of a somatic cell, a cardiac fibroblast, a non-cardiac fibroblast, a cardiac progenitor cell, and a stem cell.

16. The method of claims 1-15, wherein the reprogramming factors administered comprise Gata4, Mef2c, and Tbx5.

17. The method of claims 1-15, wherein the reprogramming factors administered comprise Myocardin, Mef2c, and Tbx5.

18. A method for generating an induced cardiomyocyte, the method comprising administering to a non-cardiomyocyte an effective amount of a WNT inhibitor, an effective amount of a TGF-β inhibitor, and reprogramming factors comprising Gata4, Mef2c, and Tbx5, thereby generating an induced cardiomyocyte.

19. A method for generating an induced cardiomyocyte, the method comprising administering to a non-cardiomyocyte an effective amount of a WNT inhibitor, an effective amount of a TGF-β inhibitor, and reprogramming factors comprising Myocardin, Mef2c, and Tbx5, thereby generating an induced cardiomyocyte.

20. The method of claims 1-19, wherein the non-cardiomyocyte is induced to a cardiomyocyte in vivo.

21. The methods of claims 1-19, further comprising administering an anti-inflammatory agent.

22. A method for generating an induced cardiomyocyte or cardiomyocyte-like cell in vivo, the method comprising: administering to a subject an effective amount of a WNT inhibitor, an effective amount of a TGF-β inhibitor, and one or more reprogramming factors, thereby generating an induced cardiomyocyte or a cardiomyocyte-like cell.

23. The method of claim 22, wherein the non-cardiomyocyte is a human cell.

24. The method of claim 22 or 23, wherein the WNT inhibitor is a small molecule.

25. The method of claim 22 or 23, wherein the WNT inhibitor is an siRNA.

26. The method of claims 22-25, wherein the TGF-β inhibitor is a small molecule.

27. The method of claims 22-25, wherein the TGF-β inhibitor is an siRNA

28. The method of claims 22-27, wherein the TGF-β inhibitor is administered prior to administration of the WNT inhibitor.

29. The method of claims 22-27, wherein the TGF-β inhibitor is administered concurrently with administration of the WNT inhibitor.

30. The method of claims 22-29, wherein the reprogramming factors comprise Gata4, Mef2c, and Tbx5.

31. The method of claims 22-29, wherein the reprogramming factors comprise Myocardin, Mef2c, and Tbx5.

32. The methods of claims 22-31, further comprising administering an anti-inflammatory agent.

33. A composition comprising a population of isolated induced cardiomyocytes produced according to the method of claims 1-21.

34. The composition of claim 33, further comprising a pharmaceutically acceptable excipient.

35. The composition of claim 33, further comprising a stabilizer and/or a preservative.

36. A substantially homogenous population of cardiomyocytes of claims 1-21.

37. A method of treating a cardiovascular disease comprising administering to a subject in need thereof an effective amount of an induced cardiomyocyte population produced by the method of any one of claims 1-21.

38. A method of treating a cardiovascular disease comprising administering to a subject in need thereof an effective amount of a WNT inhibitor, an effective amount of a TGF-β inhibitor, and one or more reprogramming factors.

39. The method of claim 38, wherein the one or more reprogramming factors comprise Myocardin, Mef2c, and Tbx5.

40. The method of claim 38, wherein the one or more reprogramming factors comprise Gata4, Mef2c, and Tbx5.

41. The method of claims 38-40, further comprising administering to the subject an effective amount of an anti-inflammatory agent
